(19) United States
(12) Patent Application Publication
(10) Pub. No.: US 2025/0326715 A1
(43) Pub. Date: Oct. 23, 2025

(54) NOVEL MAIN PROTEASE INHIBITORS, AND COMPOSITIONS AND METHODS THEREOF

(71) Applicant: University of Massachusetts, Boston, MA (US)

(72) Inventors: Paul R. Thompson, Wellesley, MA (US); Santanu Mondal, Shrewsbury, MA (US)

(21) Appl. No.: 18/870,234

(22) PCT Filed: May 7, 2023
(86) PCT No.: PCT/US2023/021281

§ 371 Date: Nov. 27, 2024

(51) Int. Cl. C07D 207/26; A61K 31/4015; A61P 31/14

(52) U.S. Cl. CPC C07D 207/26; A61K 31/4015; A61P 31/14

(57) ABSTRACT

The invention provides novel compounds that are potent inhibitors of main protease (M^{Pro}) and pharmaceutical compositions and methods thereof for treating M^{Pro}-associated or mediated diseases and conditions, such as severe acute respiratory syndrome coronavirus 2 (SARS-CoV2).

### PRIORITY CLAIMS AND RELATED APPLICATIONS

This application claims the benefit of priority to U.S. Provisional Application No. 63/347,655, filed Jun. 1, 2022, the entire content of which is incorporated herein by reference for all purposes.

### STATEMENT REGARDING FEDERALLY SPONSORED RESEARCH

This invention was made with government support under Grant no. GM118112, awarded by the National Institutes of Health. The Government has certain rights in the invention.

### TECHNICAL FIELDS OF THE INVENTION

The invention generally relates to novel compounds and therapeutic methods. More particularly, the invention relates to novel compounds that are potent inhibitors of main protease (M^{Pro}) and pharmaceutical compositions and methods thereof for treating M^{Pro}-associated or mediated diseases and conditions, such as severe acute respiratory syndrome coronavirus 2 (SARS-CoV2).

### BACKGROUND OF THE INVENTION

The worldwide impact of the coronavirus pandemic (COVID-19) on public health, safety, and economy has initiated significant research into the development of potent antivirals against SARS-CoV2. The dire need for direct acting antivirals (DAA) is highlighted by the emergence of several highly contagious SARS-CoV2 strains that can partially evade therapeutic antibodies and current vaccines. Moreover, vaccines are not effective in those with compromised immune function or recommended for those who develop anaphylaxis. These issues demonstrate the pressing need for DAAs against SARS-CoV2 and other coronaviruses that may emerge in the future. Importantly, a combination of vaccine and antiviral treatment is believed to decrease morbidity and mortality more efficiently. (DiMaio, et al. 2020 Ann. Rev. Virol. 7, iii-v; Holmes, et al. 2021 Cell 184, 4848; Hu, et al. 2021 Nat. Rev. Microbiol. 19, 141; Cameroni, et al. 2021 bioRxiv 2021.12.12.472269; Cao, et al. 2021 bioRxiv 2021.12.07.470392; Grant, et al. 2022 The Lancet Regional Health Eur. 13, 100278; Lopez, et al. 2021 N. Engl. J. Med. 385, 585; Planas, et al. 2021 bioRxiv 2021.12.14.472630; Planas, et al. 2021 Nature 596, 276.)

SARS-CoV2 is an enveloped, positive-sense, single-stranded RNA virus. The genome sequence shares ˜79% and 50% similarity to those of SARS-CoV and MERS-CoV, two other members of the betacoronavirus family that have caused major outbreaks in the past. Following internalization of the virus into host cells via angiotensin converting enzyme 2 (hACE2) receptors, the S-protein on the viral surface is cleaved by host cell proteases, including cathepsins and transmembrane serine protease 2 (TMPRSS2). Subsequent translation of the viral RNA, which encodes multiple open reading frames (ORF), generates two polyproteins, pp1a and pp1ab. These polyproteins are cleaved into 16 non-structural proteins (nsp1-16) by two ORF-encoded viral proteases, M^{Pro} and the papain-like protease (PL^{Pro}). These proteases are essential for viral protein expression, viral genome replication, virion packaging, and viral genomic RNA processing. Therefore, M^{Pro} and PL^{Pro} are promising targets to develop DAAs against SARS-CoV2. Since M^{Pro} has a broader substrate profile, it is anticipated that M^{Pro} inhibitors will lead to better therapeutic outcomes. (Chan, et al. 2012 J. Infect. 65, 477; Drosten, et al. 2003 N. Engl. J. Med. 348, 1967; Lu, et al. 2020 Lancet 395, 565; Padmanabhan, et al. 2020 PLoS Comput. Biol. 16, e1008461; Peacock, et al. 2021 Nat. Microbiol. 6, 899; Whittaker 2021 Lancet Microbe 2, e488; Gioia, et al. 2020 Biochem. Pharmacol. 182, 114225; Luan, et al. 2020 J. Proteome Res. 19, 4316; Osipiuk, et al. 2021 Nat. Commun. 12, 743.)

M^{Pro} is a cysteine protease and its active site contains a catalytic dyad comprised of C145 and H41 residues. Homodimerization of M^{Pro} is crucial for enzymatic activity as it forms the S1 pocket of the substrate-binding site (FIG. 1A). Like other betacoronaviruses, SARS-CoV2 M^{Pro} preferentially cleaves substrates with the consensus sequence: P2 (L/F/M/V), P1 (Gln), and P1′ (S/A) residues. Notably, human proteases with such substrate selectivity are rare. Since the onset of the pandemic, several M^{Pro} inhibitors have been reported and most of these covalently modify C145 with a variety of warheads, including α-ketoamides, α, β-unsaturated ketones, aldehydes, dihaloacetamides and vinyl sulfones. Recently, the US Food and Drug Administration issued an emergency use authorization for Pfizer's Paxlovid, a combination of the M^{Pro} inhibitor, nirmatrelvir and the HIV protease inhibitor ritonavir (www.ClinicalTrials.gov identifier: NCT04756531; www.fda.gov/media/155050/download). Despite the remarkable efficacy, this treatment regimen will not be accessible to all patients as several other drugs are contraindicated with ritonavir. Furthermore, Paxlovid is not recommended in patients with severe renal and/or hepatic impairment. (Jin, et al. 2020 Nature 582, 289; Zhang, et al. 2020 Science 368, 409; Dai, et al. 2020 Science 368, 1331; Hoffman, et al. 2020 J. Med. Chem. 63, 12725; Ma, et al. 2021 J Am. Chem. Soc. 143, 20697; Mengist, et al. 2021 Front. Chem. 9, 622898; Rathnayake, et al. 2020 Sci. Transl. Med. 12; Rut, et al. 2021 Nat. Chem. Biol. 17, 222; Yang, et al. 2021 ChemMedChem 16, 942; Owen, et al. 2021 Science 374, 1586.)

Furthermore, there will be the inevitable emergence of variants resistant to currently available treatments.

Therefore, an ongoing need exists for the development of novel M^{Pro} inhibitors with excellent cellular efficacy, metabolic stability, and pharmacokinetic properties.

### SUMMARY OF THE INVENTION

The invention is based in part on the unexpected discovery of novel and improved M^{Pro} inhibitors, for examples SM141 and SM142 disclosed herein, that exhibit a unique binding mode in the active site of M^{Pro}. SM141 and SM142 are completely inactive for inhibiting papain-like protease (PL^{Pro}), another cysteine protease involved in the life cycle of SARS-CoV2. SM141 and SM142 exhibit outstanding antiviral activity and block SARS-CoV2 replication in permissive epithelial cells (hACE2 expressing A549 cells) with IC₅₀ values of 8.2 and 14.7 nM. Notably, these values are not only remarkably lower than the FDA-approved M^{Pro} inhibitor, nirmatrelvir but also the lowest achieved by any M^{Pro} inhibitors developed to date.

Significantly, detailed selectivity studies indicate that SM141 and SM142 also inhibit cathepsin L (CatL), an intriguing finding as CatL cleaves the viral S protein to promote entry of the virus into host cells. These observations indicate that the antiviral activity of SM141 and SM142 results from the dual inhibition of M^{Pro} and CatL. Notably, intranasal as well as intraperitoneal administration of SM141 and SM142 lead to reduced viral replication, viral loads in the lung, and enhanced survival in SARS-CoV2 infected K18-ACE2 transgenic mice. In total, these data indicate that SM141 and SM142 represent promising scaffolds on which to develop antiviral drugs against SARS-CoV2.

In one aspect, the invention generally relates to a compound having the structural formula (I),

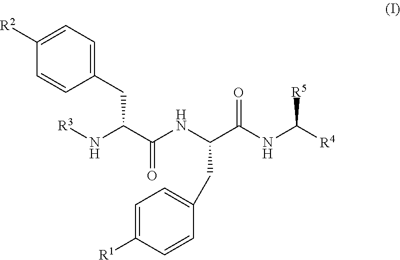

or a pharmaceutically acceptable form or an isotope derivative thereof,
wherein
R¹ is selected from the group consisting of H, F or Cl;
R² is selected from the group consisting of H, F or Cl;
R³ is selected from the group consisting of H or tert-butyloxycarbonyl (Boc) or

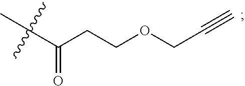 [SMILES: *C(=O)CCOCC#C]

R⁴ is

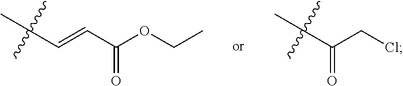

R⁵ is

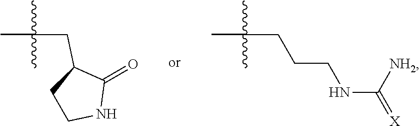

wherein X is O or NH₂⁺.

In another aspect, the invention generally relates to a pharmaceutical composition comprising a compound disclosed herein.

In yet another aspect, the invention generally relates to a pharmaceutical composition comprising a compound having the structural formula of (I):

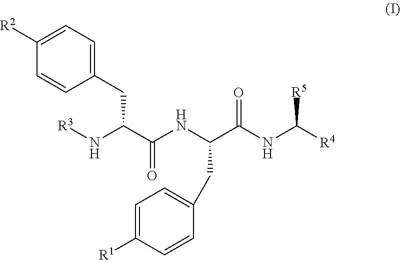

or a pharmaceutically acceptable form or an isotope derivative thereof,
wherein
R¹ is selected from the group consisting of H, F or Cl;
R² is selected from the group consisting of H, F or Cl;
R³ is selected from the group consisting of H, Boc or

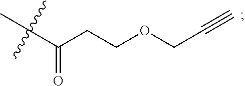 [SMILES: *C(=O)CCOCC#C]

R⁴ is

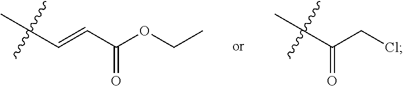

R⁵ is

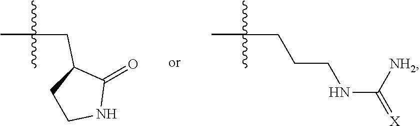

wherein X is O or NH₂⁺,
effective to treat, reduce or prevent one or more diseases or conditions, in a mammal, including a human, and a pharmaceutically acceptable excipient, carrier, or diluent.

In yet another aspect, the invention generally relates to a unit dosage form comprising a pharmaceutical composition disclosed herein.

In certain embodiments, the unit dosage form is in the form of a tablet or capsule.

In yet another aspect, the invention generally relates to a method for inhibiting or inactivating MPro in a cell, comprising contacting the cell with a compound disclosed herein.

In yet another aspect, the invention generally relates to a method for inhibiting or inactivating an activity of MPro in vitro or in vivo, comprising contacting the cell with a compound disclosed herein.

In yet another aspect, the invention generally relates to a method for treating, reducing or preventing a disease or condition, comprising administering to a subject in need thereof a compound disclosed herein.

In yet another aspect, the invention generally relates to a method for treating, reducing or preventing a disease or condition, comprising administering to a subject in need thereof a pharmaceutical composition comprising a compound having the structural formula of (I):

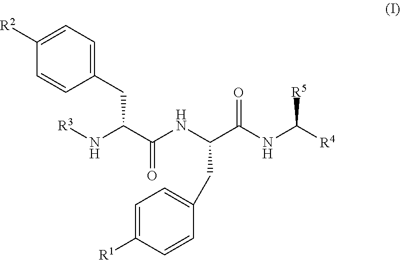

or a pharmaceutically acceptable form or an isotope derivative thereof,
wherein
R¹ is selected from the group consisting of H, F or Cl;
R² is selected from the group consisting of H, F or Cl;
R³ is selected from the group consisting of H, tert-butyloxycarbonyl (Boc) or

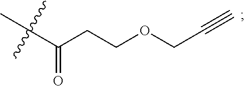 [SMILES: *C(=O)CCOCC#C]

R⁴ is

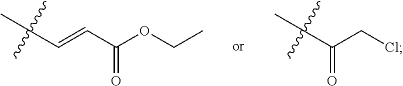

R⁵ is

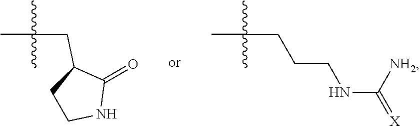

wherein X is O or NH₂⁺,
effective to treat, prevent, or reduce one or more diseases or conditions, in a mammal, including a human.

In yet another aspect, the invention generally relates to use of the compound disclosed herein, and a pharmaceutically acceptable excipient, carrier, or diluent, in preparation of a medicament for treating a disease or disorder.

### BRIEF DESCRIPTION OF THE DRAWINGS

FIG. 1. (A) Chemical structures of D-FF(R/Cit)-CMKyne and D-FF(R/Cit)-CMK. (B) M^{Pro} inhibition by D-FF(R/Cit)-CMKyne and D-FF(R/Cit)-CMK. Crystal structures of M^{Pro} in complex with D-FFR-CMKyne (C) and F-FFCit-CMKyne (D) (PDB ID: 7MAU and 7MAV). (E) Overlay of the crystal structures of M^{Pro}-D-FFR-CMKyne and M^{Pro}-D-FFR-CMK (PDB ID: 7MAT) complexes, indicating the importance of the N-terminal alkyne group for binding of the inhibitors (Carbon atoms of D-FFR-CMKyne and D-FFR-CMK are highlighted in yellow and green, respectively).

FIG. 2. (A) Chemical structures of SM136-145. Crystal structures of M^{Pro} in complex with SM141 (B, PDB ID: 7MB0), SM143 (D, PDB ID:7MB1) and SM144 (E, PDB ID: 7MB2). (C) Overlay of crystal structures of M^{Pro}-SM141 and M^{Pro}-SM137 (PDB ID: 7MAX) complexes (Carbon atoms of SM137 and SM141 are highlighted in yellow and green, respectively. Carbon atoms of the protein residues for M^{Pro}-SM137 and M^{Pro}-SM141 complexes are displayed in pink and white, respectively).

FIG. 3. Antiviral activity of the M^{Pro} inhibitors in A549-hACE2 (A) and Huh7.5 (B) cells evaluated at a concentration of 2 μM. (C) Dose-response curves for the inhibition of SARS-CoV2 infection of A549-hACE2 cells. The table indicates the corresponding EC₅₀ values.

FIG. 4. Dose-dependent labeling (A) and the limit of detection (B) of M^{Pro} by SM144. (C) Inhibition of M^{Pro} labeling by SM141. (D) Labeling of A549-hACE2 lysate spiked with recombinant M^{Pro} by SM144 and competitive inhibition with SM141 and SM142. The triangle indicates M^{Pro}. (E) Labeling of A549-hACE2 lysate by SM144 in the presence and absence of SM141 and SM142. Volcano plot indicating the proteins in the A549-hACE2 lysate enriched by SM144 in the absence (F) and presence (G) of SM141.

FIG. 5. Weight loss (A) and survival (B) of K18-ACE2 transgenic mice infected with SARS-CoV2 (3×10⁴ PFU/mouse) after intranasal treatment with SM141 or SM142 (10 mg/kg, once 2 h prior to the infection and two more doses on the consecutive days after infection). Weight loss (C) and survival (D) of K18-ACE2 transgenic mice infected with SARS-CoV-2 (3×10⁴ PFU/mouse) after intraperitoneal treatment with 25 mg/kg SM141 or SM142 twice daily for 5 days. QPCR analysis of SARS-CoV2-N(E), Nsp14 (F) and viral titer (G) in the lung tissue of K18-ACE2 transgenic mice infected with SARS-CoV2 after 72 h treatment with vehicle, SM141 or SM142. (H) Representative images of H&E-stained lung sections from SARS-CoV2-infected K18-ACE2 transgenic mice receiving intraperitoneal injection of SM141, SM142 or vehicle for 5 days.

FIG. 6. (A) Chemical structures of D-FF(R/Cit)-(CMK/CMkynes). (B) SARS-CoV-2 M^{Pro} Inhibition by D-FF(R/Cit)-(CK/CMkynes) ([I]=0-3.3 μM; [M^{Pro} ]=12.5 nM, [Substrate]=30 μM). (C and D) Time-dependent inactivation of SARS-CoV-2 M^{Pro} by FFR-CMKyne (C) and FFCit-CMkyne (D): 0, 13, 25, 50, 100 nM (top to bottom) showing single phase association kinetics ([I]=0-100 nM; [M^{Pro}]=12.5 nM, [Substrate]=30 μM). (E) Dose-dependent inactivation of SARS-CoV-2 M^{Pro} by FFR-CMKyne and FFCit-CMkyne.

FIG. 7. SARS-CoV-2 PL^{Pro} inhibition by D-FFR-(CMK/CMKyne) (A) and D-FFCit-(CMK/CMKyne) (B).

FIG. 8. ¹H (A) and ¹³C (B) NMR spectra of SM136 in d₆-DMSO.

FIG. 9. HPLC trace (A) and ESI-Mass spectra (B) of SM136.

FIG. 10. ¹H (A) and ¹³C (B) NMR spectra of SM137 in d₆-DMSO.

FIG. 11. HPLC trace (A) and ESI-Mass spectra (B) of SM137.

FIG. 12. ¹H (A) and ¹³C (B) NMR spectra of SM138 in d₆-DMSO.

FIG. 13. HPLC trace (A) and ESI-Mass spectra (B) of SM138.

FIG. 14. ¹H (A) and ¹³C (B) NMR spectra of SM139 in d₆-DMSO.

FIG. 15. HPLC trace (A) and ESI-Mass spectra (B) of SM139.

FIG. 16. ¹H (A) and ¹³C (B) NMR spectra of SM140 in d₆-DMSO.

FIG. 17. HPLC trace (A) and ESI-Mass spectra (B) of SM140.

FIG. 18. ¹H (A) and ¹³C (B) NMR spectra of SM141 in d₆-DMSO.

FIG. 19. HPLC trace (A) and ESI-Mass spectra (B) of SM141.

FIG. 20. ¹H (A) and ¹³C (B) NMR spectra of SM142 in d₆-DMSO.

FIG. 21. HPLC trace (A) and ESI-Mass spectra (B) of SM142.

FIG. 22. ¹H (A) and ¹³C (B) NMR spectra of SM143 in d₆-DMSO.

FIG. 23. HPLC trace (A) and ESI-Mass spectra (B) of SM143.

FIG. 24. ¹H (A) and ¹³C (B) NMR spectra of SM144 in d₆-DMSO.

FIG. 25. HPLC trace (A) and ESI-Mass spectra (B) of SM144.

FIG. 26. ¹H (A) and ¹³C (B) NMR spectra of SM145 in d₆-DMSO.

FIG. 27. HPLC trace (A) and ESI-Mass spectra (B) of SM145.

FIG. 28. (A) Chemical Structures of SM136 and SM137. (B) SARS-CoV-2 M^{Pro} Inhibition by SM136 and 137. (C and D) Time-dependent inactivation of SARS-CoV-2 M^{Pro} by SM136 (C) and SM137 (D), [I]=0, 6.2, 12.5, 25, 50 μM (top to bottom) showing single phase association kinetics. (E) Dose-dependent inactivation of SARS-CoV-2 M^{Pro} by SM136 and SM137. The quoted uncertainty in these kinetic parameters is the standard deviation from the triplicate measurements of these data.

FIG. 29. (A) Chemical structures of SM138 and SM139. (B) SARS-CoV-2 M^{Pro} Inhibition by SM138 and SM139. (C and D) Time-dependent inactivation of SARS-CoV-2 M^{Pro} by SM138 (C) and SM139 (D), [I]=0, 6.2, 12.5, 25, 50 μM (top to bottom) showing single phase association kinetics. (E) Dose-dependent inactivation of SARS-CoV-2 M^{Pro} by SM138 and SM139. The quoted uncertainty in these kinetic parameters is the standard deviation from the triplicate measurements of these data.

FIG. 30. (A) Chemical structures of SM140 and SM141. (B) SARS-CoV-2 M^{Pro} Inhibition by SM140 and SM141. (C and D) Time-dependent inactivation of SARS-CoV-2 M^{Pro} by SM140 (C) and SM141 (D), [I]=0, 3.1, 6.2, 12.5, 12.5, 25 μM (top to bottom) showing single phase association kinetics. (E) Dose-dependent inactivation of SARS-CoV-2 M^{Pro} by SM140 and SM141. The quoted uncertainty in these kinetic parameters is the standard deviation from the triplicate measurements of these data.

FIG. 31. (A) Chemical structures of SM142 and SM143. (B) SARS-CoV-2 M^{Pro} Inhibition by SM142 and SM143. (C and D) Time-dependent inactivation of SARS-CoV-2 M^{Pro} by SM142 (C) and SM143 (D), [I]=0, 1.5, 3.1, 6.2, 12.5, 12.5 μM (top to bottom) showing single phase association kinetics. (E) Dose-dependent inactivation of SARS-CoV-2 M^{Pro} by SM142 and SM143. The quoted uncertainty in these kinetic parameters is the standard deviation from the triplicate measurements of these data.

FIG. 32. (A) Chemical structure of SM144. (B and C) SARS-CoV-2 M^{Pro} Inhibition by SM144 (B: IC₅₀, C: Kᵢ). (D) Time-dependent inactivation of SARS-CoV-2 M^{Pro} by SM144, [I]=0, 0.75, 1.5, 3.1, 6.2, 12.5 M (top to bottom) showing single phase association kinetics. (E) Dose-dependent inactivation of SARS-CoV-2 M^{Pro} by SM144. The quoted uncertainty in these kinetic parameters is the standard deviation from the measurements of these data.

FIG. 33. (A) Chemical structure of SM145. (B and C) SARS-CoV-2 M^{Pro} Inhibition by SM145 (B: IC₅₀, C: Kᵢ). (D) Time-dependent inactivation of SARS-CoV-2 M^{Pro} by SM145, [I]=0, 0.75, 1.5, 3.1, 6.2, 12.5 M (top to bottom) showing single phase association kinetics. (E) Dose-dependent inactivation of SARS-CoV-2 M^{Pro} by SM145. The quoted uncertainty in these kinetic parameters is the standard deviation from the triplicate measurements of these data.

FIG. 34. Crystal Structure of SARS-CoV-2 M^{Pro} in complex with SM137 (A), SM139 (B), SM141 (C), SM143 (D), SM144 (E) and SM145 (F), indicating the residues that contribute to the binding of the inhibitors.

FIG. 35. (A) Cytotoxicity of SM140-145 and D-FF(R/Cit)-CMKyne. A549-hACE2 cells were treated with indicated doses of inhibitors for 24 hours. Then the media were collected for lactate dehydrogenase (LDH) assay. (B) qRT-PCR analysis of OC-43 virus mRNA expression in A549 cells treated with indicated compounds.

FIG. 36. Dose-dependent inhibition of cathepsin L (A) and cathepsin B (B) by SM141-145. IC₅₀ values are given in Table 1. These data were generated using commercially available cathepsin L inhibition assay kits (Abeam).

FIG. 37. Time- (Left panel) and concentration-dependent (right panel) inactivation of cathepsin L by SM141-145. These data were generated using commercially available cathepsin L inhibition assay kits (Abcam).

FIG. 38. (A) Weight change of mock-infected mice independently treated with PBS, SM141 and SM142. (B-E) QPCR analysis of representative cytokines including IFNb, TNF, IL1b and IL6 in lung tissue of K18-ACE2 transgenic mice infected with SARS-CoV-2 for 72 hours with indicated treatments.

### DEFINITIONS

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

As used herein, “administration” of a disclosed compound encompasses the delivery to a subject of a compound as described herein, or a prodrug or other pharmaceutically acceptable derivative thereof, using any suitable formulation or route of administration, as discussed herein.

As used herein, the terms “effective amount” or “therapeutically effective amount” refer to that amount of a compound or pharmaceutical composition described herein that is sufficient to effect the intended application including, but not limited to, disease treatment, as illustrated below.

In some embodiments, the amount that is effective to stop the progression or effect reduction of a disease or disorder associated with SARS-CoV2 infections.

The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. The term also applies to a dose that will induce a particular response in target cells, e.g., reduction of cell migration. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. Treatment is aimed to obtain beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a particular disease, or to a patient reporting one or more of the physiological symptoms of a disease, even though a diagnosis of this disease may not have been made. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

As used herein, the term “therapeutic effect” refers to a therapeutic benefit and/or a prophylactic benefit as described herein. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, a “pharmaceutically acceptable form” of a disclosed compound includes, but is not limited to, pharmaceutically acceptable salts, esters, hydrates, solvates, isomers, prodrugs, and isotopically labeled derivatives of disclosed compounds. In one embodiment, a “pharmaceutically acceptable form” includes, but is not limited to, pharmaceutically acceptable salts, esters, isomers, prodrugs and isotopically labeled derivatives of disclosed compounds. In some embodiments, a “pharmaceutically acceptable form” includes, but is not limited to, pharmaceutically acceptable salts, esters, stereoisomers, prodrugs and isotopically labeled derivatives of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)⁴ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a pharmaceutically acceptable ester. As used herein, the term “pharmaceutically acceptable ester” refers to esters that hydrolyze in vivo and include those that break down readily in the human body to leave the parent compound or a salt thereof. Such esters can act as a prodrug as defined herein. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfinic acids, sulfonic acids and boronic acids. Examples of esters include formates, acetates, propionates, butyrates, acrylates and ethylsuccinates. The esters can be formed with a hydroxy or carboxylic acid group of the parent compound.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (e.g., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

### DETAILED DESCRIPTION OF THE INVENTION

The invention provides novel and potent inhibitors of M^{Pro} and methods for treating M^{Pro}-associated or mediated diseases and conditions, such as severe acute respiratory syndrome coronavirus 2 (SARS-CoV2).

With the identification of a novel D-FF motif that exhibits a unique binding mode in the active site of M^{Pro} when conjugated with an electrophilic cysteine-targeted warhead at the C-terminus, novel compounds are disclosed herein that inhibit M^{Pro} with nanomolar potency. For examples, SM141 and SM142 inhibit the replication of SARS-CoV2 in A549 cells expressing human ACE2 receptor with IC₅₀ values of 8.2 and 14.7 nM, respectively. Such remarkable cellular antiviral efficacy was shown to result from the combined inhibition of M^{Pro} and CatL by these inhibitors. Additionally, intraperitoneal administration of SM141 and SM142 into SARS-CoV2 infected K18-hACE2 mice markedly inhibits viral replication as well as reduces lung damage and inflammation, indicating that these compounds are potential candidates for the development of antivirals against SARS-CoV2.

In one aspect, the invention generally relates to a compound having the structural formula (I),

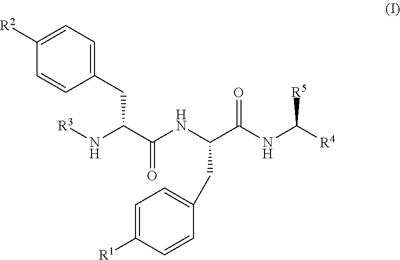

or a pharmaceutically acceptable form or an isotope derivative thereof,
wherein
R¹ is selected from the group consisting of H, F or Cl;
R² is selected from the group consisting of H, F or Cl;
R³ is selected from the group consisting of H or Boc or

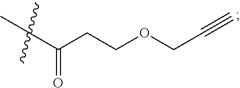 [SMILES: *C(=O)CCOCC#C]

R⁴ is

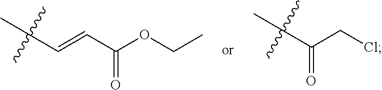

R⁵ is

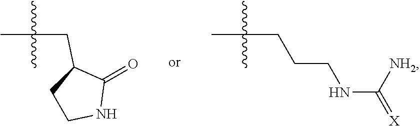

wherein X is O or NH₂⁺.

In certain embodiments,
R⁴ is

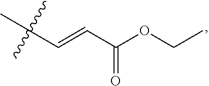 [SMILES: */C=C/C(=O)OCC]

and
R⁵ is

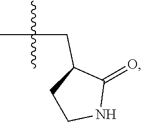 [SMILES: *C[C@@H]1CCNC1=O]

with the compound having the structural formula:

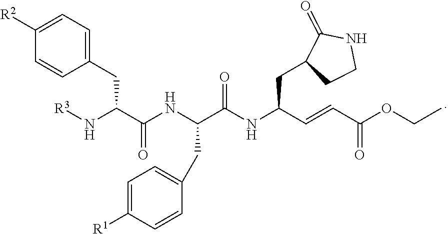

In certain embodiments, R¹ is H.

In certain embodiments, R¹ is F.

In certain embodiments, R² is H.

In certain embodiments, R² is F.

In certain embodiments, R³ is H.

In certain embodiments, R³ is Boc.

In certain embodiments, R³ is

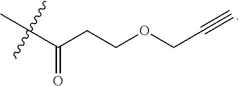 [SMILES: *C(=O)CCOCC#C]

Exemplary compounds of the invention include:

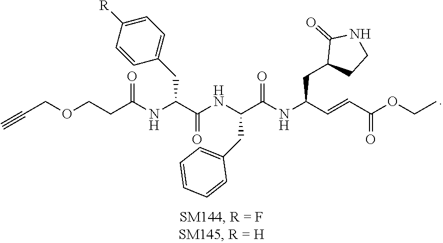

Exemplary compounds of the invention also include:

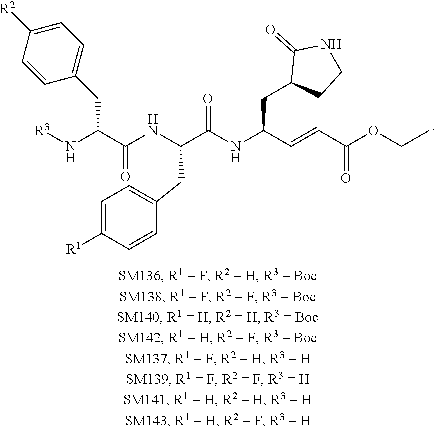

In another aspect, the invention generally relates to a pharmaceutical composition comprising a compound disclosed herein.

In yet another aspect, the invention generally relates to a pharmaceutical composition comprising a compound having the structural formula of (I):

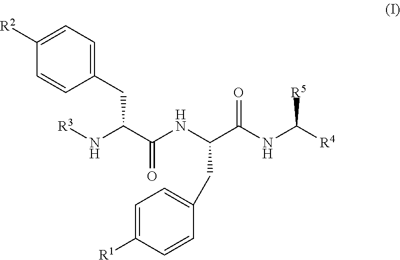

or a pharmaceutically acceptable form or an isotope derivative thereof,
wherein
R¹ is selected from the group consisting of H, F or Cl;
R² is selected from the group consisting of H, F or Cl;
R³ is selected from the group consisting of H, Boc or

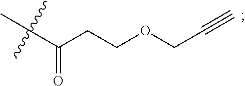 [SMILES: *C(=O)CCOCC#C]

R⁴ is

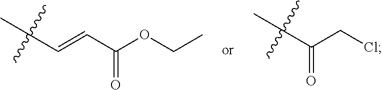

R⁵ is

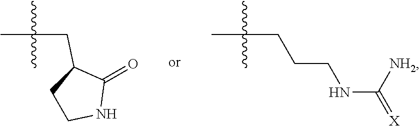

wherein X is O or NH₂⁺,
effective to treat, reduce or prevent one or more diseases or conditions, in a mammal, including a human, and a pharmaceutically acceptable excipient, carrier, or diluent.

In certain embodiments, the one or more diseases or conditions is SARS-CoV2 or a related disease or condition.

In yet another aspect, the invention generally relates to a unit dosage form comprising a pharmaceutical composition disclosed herein.

In certain embodiments, the unit dosage form is in the form of a tablet or capsule.

In yet another aspect, the invention generally relates to a method for inhibiting or inactivating MPro in a cell, comprising contacting the cell with a compound disclosed herein.

In yet another aspect, the invention generally relates to a method for inhibiting or inactivating an activity of MPro in vitro or in vivo, comprising contacting the cell with a compound disclosed herein.

In yet another aspect, the invention generally relates to a method for treating, reducing or preventing a disease or condition, comprising administering to a subject in need thereof a compound disclosed herein.

In yet another aspect, the invention generally relates to a method for treating, reducing or preventing a disease or condition, comprising administering to a subject in need thereof a pharmaceutical composition comprising a compound having the structural formula of (I):

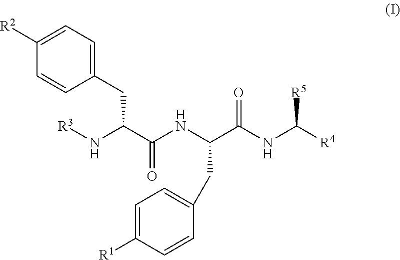

or a pharmaceutically acceptable form or an isotope derivative thereof,
wherein
R¹ is selected from the group consisting of H, F or Cl;
   R² is selected from the group consisting of H, F or Cl;
   R³ is selected from the group consisting of H, tert-butyloxycarbonyl (Boc) or

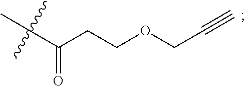 [SMILES: *C(=O)CCOCC#C]

R⁴ is

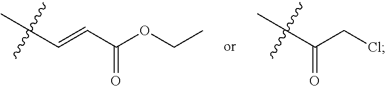

R⁵ is

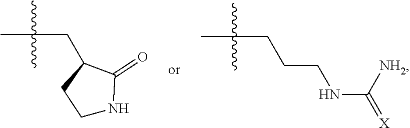

wherein X is O or NH₂⁺,
effective to treat, prevent, or reduce one or more diseases or conditions, in a mammal, including a human.

In certain embodiments, the one or more diseases or conditions is mediated by or associated with an activity of MPro.

In certain embodiments, the one or more diseases or conditions is SARS-CoV2 or a related disease or condition.

In yet another aspect, the invention generally relates to use of the compound disclosed herein, and a pharmaceutically acceptable excipient, carder, or diluent, in preparation of a medicament for treating a disease or disorder.

In certain embodiments, the one or more diseases or conditions is SARS-CoV2 or a related disease or condition.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure. Solvates and polymorphs of the compounds of the invention are also contemplated herein. Solvates of the compounds of the present invention include, for example, hydrates.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

The below Examples describe certain exemplary embodiments of compounds prepared according to the disclosed invention. It will be appreciated that the following general methods, and other methods known to one of ordinary skill in the art, can be applied to compounds and subclasses and species thereof, as disclosed herein.

### EXAMPLES

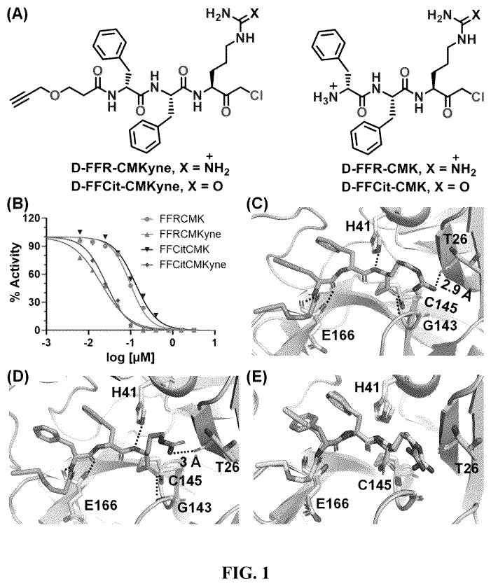

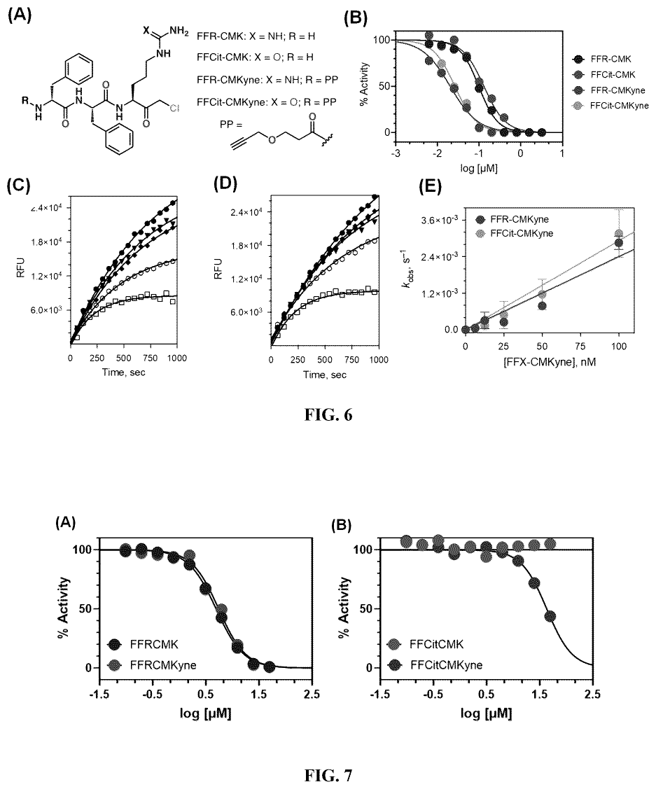

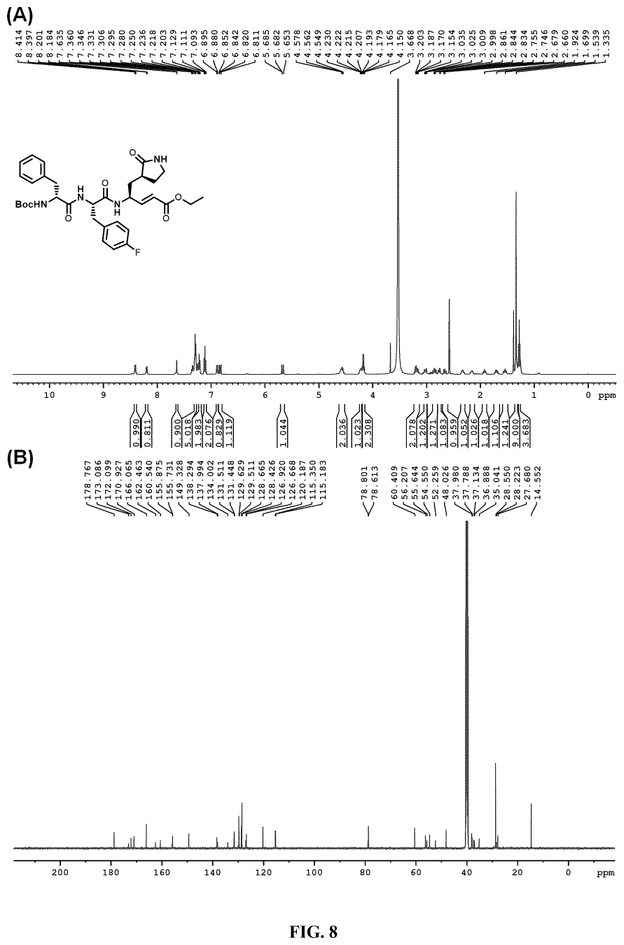

To identify potent M^{Pro} inhibitors, a library of FDA-approved drugs, covalent fragments and an in-house collection of compounds were screened. Because several proteases capable of cleaving after Gln (e.g., cathepsin B) also cleave after citrulline (Cit) (Thompson lab unpublished data), several probes that were initially developed to identify proteases that cleave after Cit were included in the screens. Interestingly, these probes, D-FFR-CMKyne and D-FFCit-CMKyne, were the top hits from the screen. These compounds contain a C-terminal arginine or Cit followed by a cysteine-reactive chloromethyl ketone (CMK) warhead (FIG. 1A). Additionally, they possess L-Phe and D-Phe residues at the P2 and P3 positions, respectively. The N-terminus of the probes contains an aliphatic alkyne handle to enable affinity enrichment of proteins via click chemistry. Gratifyingly, D-FFR-CMKyne and D-FFCit-CMKyne exhibited impressive biochemical IC₅₀ values of 20±2 and 25±5 nM, respectively, for the inhibition of M^{Pro} (FIG. 1B, Table 1). Probes lacking the alkyne, D-FFR-CMK and D-FFCit-CMK, are 4-5-fold less potent inhibitors than their parents, indicating that the N-terminal capping group substantially increases the binding affinity of these molecules to M^{Pro}. Since these molecules contain a reactive electrophilic warhead, time-dependent inhibition experiments were performed. It was found that the second-order rate constants for enzyme inactivation (k_{inact}/K_{I}) for D-FFR-CMKyne and D-FFCit-CMKyne are (1.6±0.1)×10⁶ and (1.9±0.2)×10⁶ M⁻¹ min⁻¹, respectively (Table 1, FIG. 6). These rapid inactivation rates are consistent with the potent IC₅₀ values. Although saturation was not observed in the k_{obs} versus [I] plots, likely due to a slow chemical step of inactivation, it was possible to estimate K_{I} from the initial portion of the progress curves. Notably, D-FFR-CMKyne and D-FFCit-CMKyne possess K_{I} values of 300 and 200 nM, respectively. Moreover, both D-FFR-CMKyne and D-FFCit-CMKyne are more than 275-fold selective for M^{Pro} over PL^{Pro} (Table 1 and FIG. 7).

Crystal structures of these inhibitors in complex with M^{Pro} (Table 4) confirm that covalent bond formation occurred between Cys145 and the CMK warhead. The carbonyl on the CMK warhead H-bonds with the backbone amide of Cys145 and Gly143, likely positioning the warhead for nucleophilic attack (FIGS. 1C, D). Notably, the Arg and Cit at the P1 position of D-FFR-CMKyne and D-FFCit-CMKyne occupy the S1′ pocket of the substrate-binding site, make extensive van der Waals packing with Gly143 and form H-bonds with the backbone carbonyl on Thr26 (FIGS. 1C, D). These inhibitors do not occupy the S1 site which stands in stark contrast to most inhibitors that utilize the γ-lactam mimic of a Gln, which occupies the S1 site in M^{Pro}. (Jin, et al. 2020 Nature 582, 289; Zhang, et al. 2020 Science 368, 409; Hoffman, et al. 2020 J. Med. Chem. 63, 12725; Lockbaum, et al. 2021 Biochemistry 60, 2925; Zaidman, et al. 2021 Cell Chem. Biol. 28, 1795; Zhang, et al. 2021 ACS Cent. Sci. 7, 467.)

Owing to the D-configuration of the P3 Phe, the P2 and P3 phenylalanines are both directed towards the same side of the inhibitor. In this orientation, the two phenyl rings form a π-πstacking interaction, and interact with 187-192 loop which covers the S2-S4 pockets. This binding mode is also supported by several H-bonds between the P3 Phe and the backbone of Glu166 and between the P1 amide and the side chain of the catalytic His41. Notably, removal of the alkyne handle would impact the H-bonds between the P3 Phe and Glu166 (FIG. 1E), consistent with the 4-5-fold loss in potency.

Since CMK is an extremely reactive warhead and often leads to significant off-target toxicity, developing inhibitors with a less reactive warhead followed.

TABLE 1
Steady-state kinetic inhibition parameters of the Inhibitors

| | M^{Pro} | | PL^{Pro} | | Cathepsin L | | Cathepsin B |
|---|---|---|---|---|---|---|---|
| | IC₅₀ | k_{inact}/K_{I} | Kᵢ | IC₅₀ | IC₅₀ | k_{inact}/K_{I} | IC₅₀ |
| Inhibitor | (μM) | (M⁻¹min⁻¹) | (μM) | (μM) | (nM) | (M⁻¹min⁻¹) | (μM) |
| D-FFR- | 0.020 ± | (1.6 ± | 0.3 ± | 5.5 ± | — | — | — |
| CMKyne | 0.002 | 0.1) × 10⁶ | 0.04 | 0.7 | | | |
| D-FFCit- | 0.025 ± | (1.9 ± | 0.2 ± | 40 ± | — | — | — |
| CMKyne | 0.005 | 0.2) × 10⁶ | 0.05 | 5 | | | |
| D-FFR- | 0.10 ± | — | — | 5 ± | — | — | — |
| CMK | 0.010 | | | 0.3 | | | |
| D-FFCit- | 0.13 ± | — | — | >50 | — | — | — |
| CMK | 0.015 | | | | | | |
| SM136 | 20 ± | (7.7 ± | 73 ± | — | — | — | — |
| | 3 | 0.3) × 10² | 4 | | | | |
| SM138 | 23 ± | (6.5 ± | 92 ± | — | — | — | — |
| | 2 | 0.2) × 10² | 14 | | | | |
| SM140 | 2.2 ± | (9.0 ± | 16 ± | >50 | — | — | >50 |
| | 0.2 | 0.2) × 10³ | 2 | | | | |
| SM142 | 1.8 ± | (1.1 ± | 10 ± | >50 | 145 ± | (1.1 ± | 40 ± 9 |
| | 0.1 | 0.3) × 10⁴ | 1 | | 14 | 0.01) × 10⁵ | |
| SM137 | 10 ± | (1.8 ± | 50 ± | — | — | — | — |
| | 2 | 0.2) × 10³ | 6 | | | | |
| SM139 | 10 ± | (1.5 ± | 65 ± | — | — | — | — |
| | 1 | 0.1) × 10³ | 5 | | | | |
| SM141 | 0.9 ± | (2.0 ± | 9 ± | >50 | 60 ± | (1.9 ± | >50 |
| | 0.1 | 0.2) × 10⁴ | 1 | | 1 | 0.5) × 10⁵ | |
| SM143 | 0.9 ± | (2.1 ± | 7 ± | >50 | 73 ± | (1.4 ± | >50 |
| | 0.1 | 0.1) × 10⁴ | 1 | | 3 | 0.4) × 10⁵ | |
| SM144 | 0.7 ± | (3.4 ± | 4.5 ± | >50 | 220 ± | (6 ± | >50 |
| | 0.1 | 0.1) × 10⁴ | 0.5 | | 26 | 0.2) × 10⁴ | |
| SM145 | 0.8 ± | (2.4 ± | 5.3 ± | >50 | 200 ± | (2.5 ± | >50 |
| | 0.1 | 0.1) × 10⁴ | 0.4 | | 4 | 0.01) × 10⁴ | |

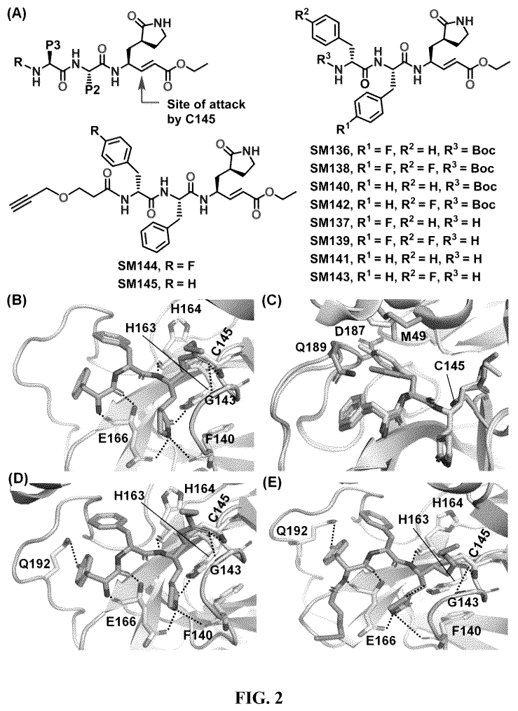

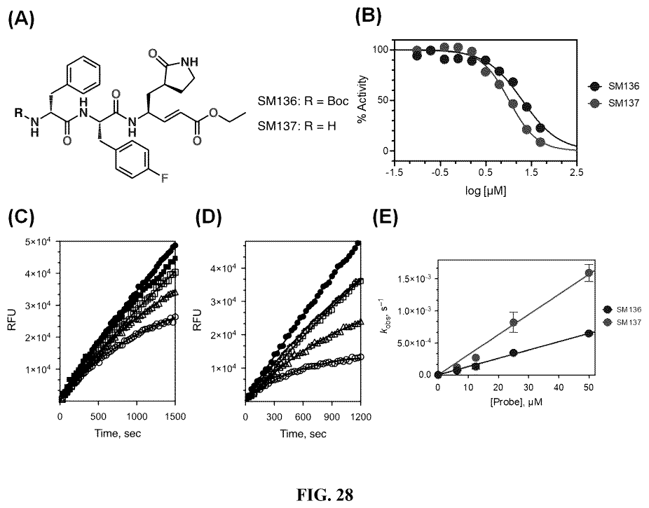

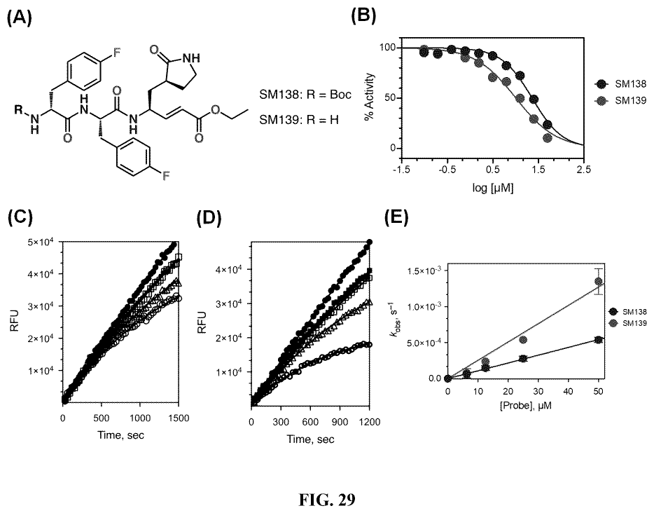

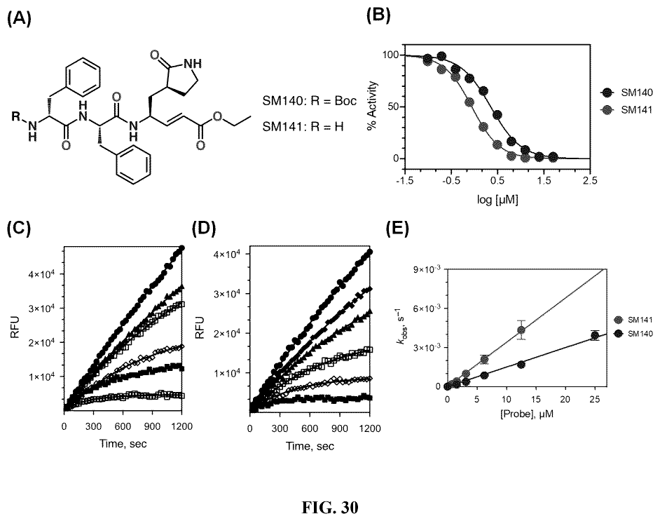

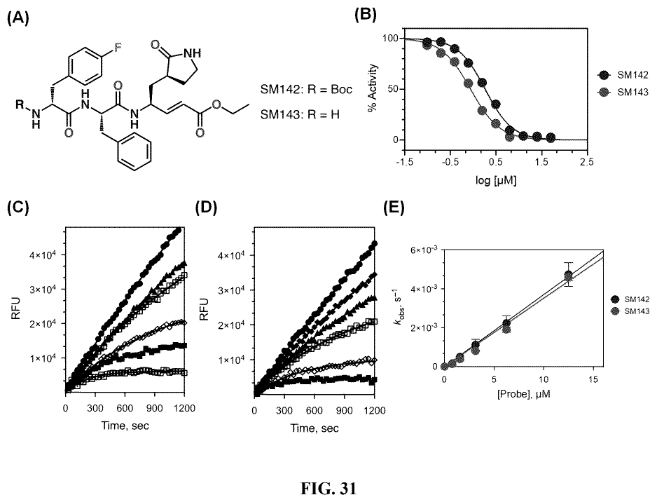

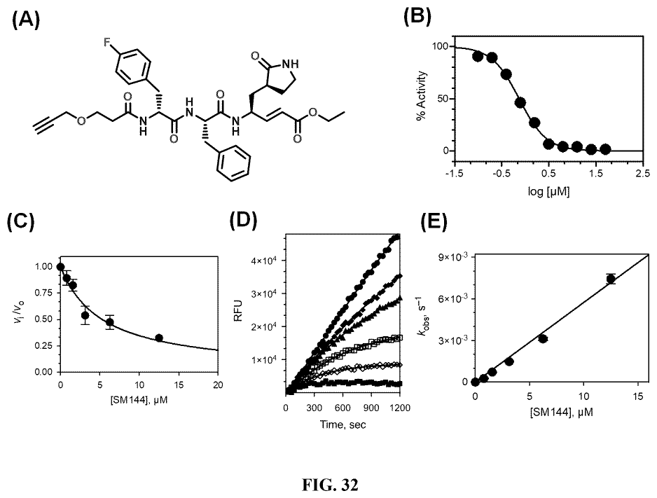

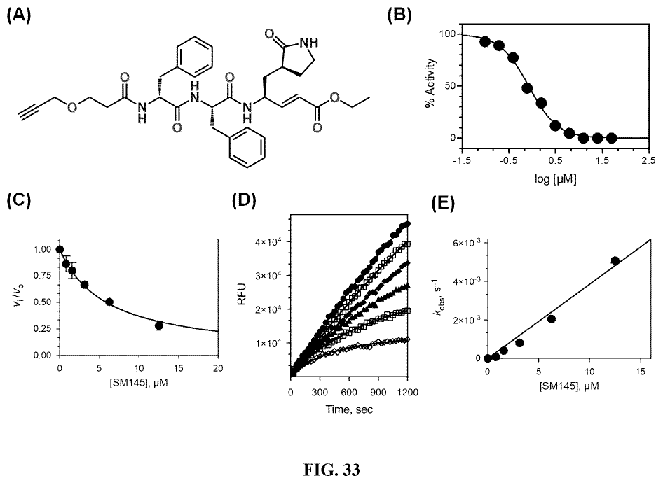

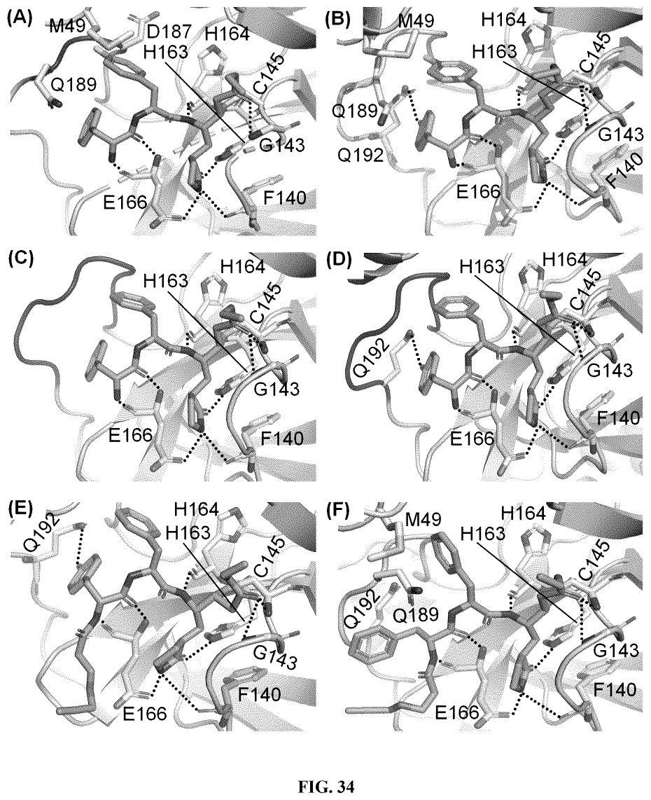

M^{Pro} prefers Gln at the P1 position, most of the M^{Pro} covalent inhibitors reported in literature contain a γ-lactam mimic of Gln that binds in the S1 pocket. To generate a hybrid M^{Pro} inhibitor, the D-Phe-Phe motif was conjugated to a γ-lactam-derived acrylate warhead to generate a series of such inhibitors (FIGS. 2A and S3-22). Since the S2 and S4 pockets of M^{Pro} accommodate hydrophobic groups, compounds containing 4-fluorophenylalanine and/or 4-fluoro-D-phenylalanine at the P2 and P3 positions were synthesized. Also synthesized were compounds that have an N-terminal Boc group as well as aliphatic alkyne functionality like the parent compounds. The 4-fluoro substitution on the P2 residue does not seem to be well accommodated in the active site of M^{Pro}, while the same on P3 residue affords fairly potent inhibitors (Table 1). Interestingly, the presence of the N-terminal Boc group significantly diminishes the potency, while the most potent inhibitors in this series, SM144 (IC₅₀=0.7 μM; k_{inact}/K_{I}=(3.4±0.1)×10⁴ M⁻¹ min⁻¹) and SM145 (IC₅₀=0.8 μM; k_{inact}/K_{I}=(2.4±0.1)×10⁴ M⁻¹ min⁻¹) possess the aliphatic alkyne handle at the N-terminus (Table 1 and FIGS. 28-33). Notably, none of these compounds inhibit PL^{Pro} at up to 50 μM concentration, highlighting their excellent selectivity to M^{Pro}.

Co-crystal structures (Table 4) showed that all inhibitors form a thioether bond with the active site cysteine (C145), confirming the covalent nature of their inhibition. Similar to other known M^{Pro} covalent inhibitors, the γ-lactam mimetic of glutamine occupies the S1 pocket of the substrate-binding site (FIGS. 2B-E and S29). As observed for D-FF(R/Cit)-CMKynes, the amide and carbonyl groups of the P3 residue H-bond with the backbone carbonyl and amide groups of Glu166. Also, the P1 amide moiety forms an H-bond with His164 (FIGS. 2B-E). Interestingly, the two aromatic groups at P2 and P3 positions show “T” edge-to-face stacking rather than face-to-face stacking as observed for D-FFR-CMKyne and D-FFCit-CMKyne. Overlays of SM141 and SM137 show that the 4-fluoro-Phe at the P2 position clashes with the loop between residues D186-Q188 and M49, accounting for the poor activity of SM137 and SM139 (FIG. 2C). However, the fluorine on 4-fluoro-Phe at the P3 of SM143 interacts with Gln192, likely explaining why it retains potency (FIG. 2D). The N-terminal alkyne handle forms additional favorable interactions (FIG. 2E), although it packs in a different conformation than in the structures of M^{Pro} bound to the D-FF(R/Cit)CMKynes (FIGS. 1C, D).

Given the nanomolar potency of these inhibitors, their antiviral efficacy was tested in mammalian cells infected with SARS-CoV2. Independently treated were human adenocarcinoma-derived alveolar basal epithelial A549 cells constitutively expressing hACE2 (A549-hACE2) and human hepatocellular carcinoma Huh7.5 cells with SARS-CoV2 at a multiplicity of infection (MOI) 0.05 for 1 h to allow for viral entry. (Humphries, et al. 2021 Sci. Immunol. 6, eabi9002.)

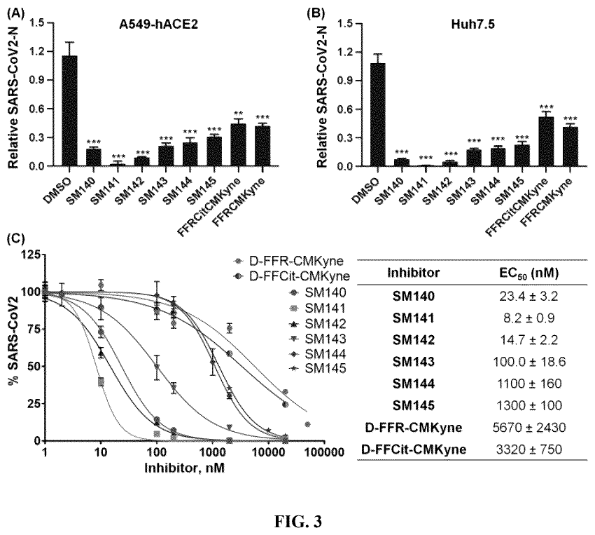

Cells were then treated with an M^{Pro} inhibitor (2 μM final) for 24 h. Total RNA in the cells was then extracted, reverse-transcribed using the iScript cDNA synthesis kit (Bio-Rad) and diluted cDNAs were subjected to qPCR analysis using iQ SYBR Green Supermix reagent (Bio-Rad). Gene expression levels of SARS-CoV-2 mRNA were normalized to GAPDH, a housekeeping gene. In both cell lines, D-FFR-CMKyne and D-FFCit-CMKyne exhibit poor antiviral activity, likely due to the higher reactivity of the CMK warhead that engages multiple off-targets (FIGS. 3A and B). By contrast, the hybrid inhibitors exhibited significantly higher antiviral efficacy with SM141 being the most potent. This compound almost completely blocks viral infection in both the cell types at a concentration of 2 μM (FIGS. 3A and B). Intrigued by these results, a dose-response study in A549-hACE2 cells (FIG. 3C) was conducted. SM141 exhibits remarkable EC₅₀ and EC₉₀ values of 8.2 and 22.1 nM, respectively, for the inhibition of SARS-CoV-2 infection. These values are remarkably better than nirmatrelvir/PF-07331332, a reversible covalent M^{Pro} inhibitor that blocks SARS-CoV2 infection in A549-hACE2 cells with EC₅₀ and EC₉₀ values of 77.9 and 215 nM, respectively. (See Table 2.)

TABLE 2
Comparison of SM141 to nirmatrelvir (PF07331332)

| | |
|---|---|
| SM141 | PF07331332^{a} |

| | | | | |
|---|---|---|---|---|
| EC₅₀ | 8.2 | nM | 78 | nM |
| EC₉₀ | 22 | nM | 215 | nM |

| | | |
|---|---|---|
| Fₐ × F_{g} | nd | 95% |

| | | | | |
|---|---|---|---|---|
| Mouse dose | 10 | mg/kg qd | >300 | m/kg bid |
| cytoxcity | >100 | μM | >3 | μM |

| | | |
|---|---|---|
| CYP3A4/5 | nd | HIGH |
| metabolism | | |

^{a}Data from medRxiv preprint doi::https://doi.org/10.1101/2021.07.28.21261232;
qd = once daily;
bid = twice daily;

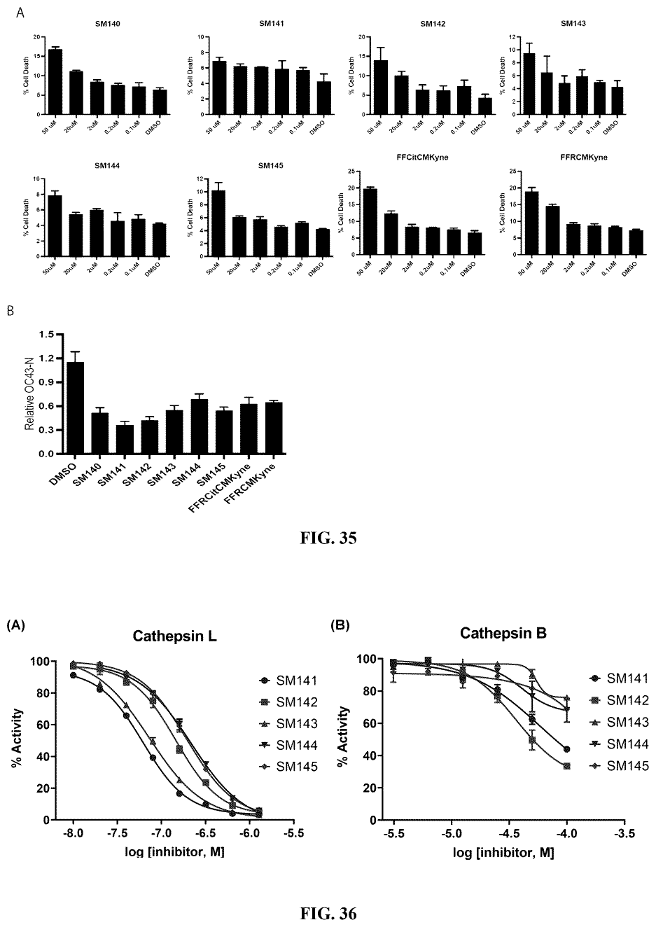

Importantly, SM141 does not cause any notable cytotoxicity up to 50 μM, whereas this concentration is only 3 M for nirmatrelvir (FIG. 35A). As such, SM141 is currently the most potent antiviral developed against SARS-CoV2. SM142 also significantly blocks SARS-CoV2 infection in A549-hACE2 cells with an EC₅₀ of 14.7 nM. Notably, the somewhat more potent hybrid inhibitors, SM144 and SM145, exhibited significantly lower antiviral activity than SM141 and SM142. These results indicate that the N-terminal aliphatic alkyne functionality in SM144 and SM145 may facilitate faster metabolism and/or efflux of these compounds, resulting in the poor antiviral activity. Although to a lesser extent than SARS-CoV2, SM141 and SM142 also inhibit OC-43, another member of the beta coronavirus family in A549-hACE2 cells (FIG. 35B).

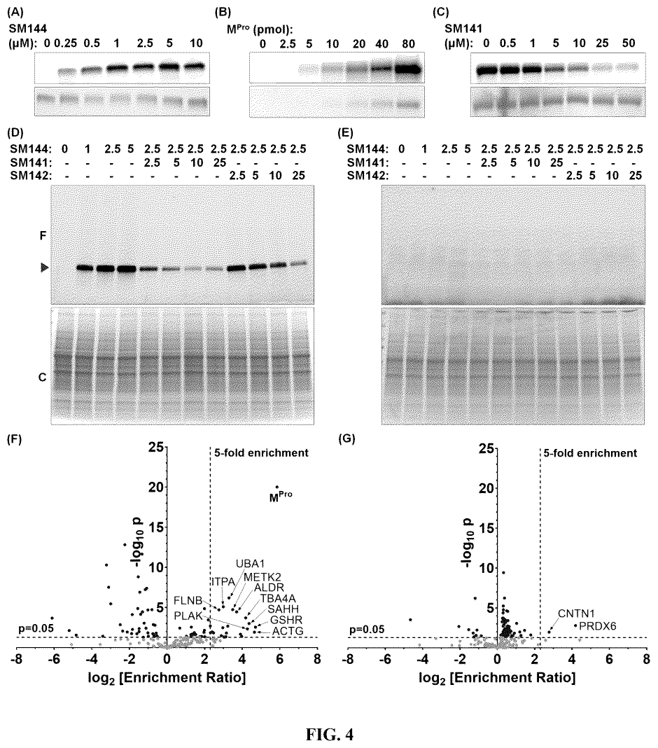

Although SM141 and SM142 exhibit remarkable antiviral activity in cells, the corresponding EC₅₀ values are more than 100-fold lower than their potency for the M^{Pro} inhibition. These observations suggested that SM141 and SM142 may target multiple proteins in A549-hACE2 cells that synergize with the inhibition of M^{Pro}. Since SM144 possesses an alkyne functionality and covalently modifies M^{Pro}, it represents an activity-based probe that can be coupled to reporter tags (e.g., TAMRA-azide or biotin-azide) using copper-catalyzed azide-alkyne click chemistry. Therefore, SM144 can be utilized to enrich its cellular targets on agarose beads and subsequently identify those proteins via chemoproteomic approaches. Notably, treatment of M^{Pro} with SM144 followed by copper-catalyzed click chemistry in the presence of TAMRA-azide and visualization by in-gel fluorescence indicated that SM144 can dose-dependently label recombinant M^{Pro} (FIG. 4A). The limit of detection of M^{Pro} by SM144 was found to be 5 pmol. Furthermore, labeling of recombinant M^{Pro} by SM144 is competitively inhibited by SM141, confirming the engagement of M^{Pro} active site by both these molecules (FIG. 4C). Next, SM144 was used to evaluate its selectivity for M^{Pro} in the presence of a complex proteome. For safety concerns, putative targets were identified by spiking the A549-hACE2 lysates with recombinant M^{Pro} (250 nM) rather than using SARS-CoV2 infected cells. Importantly, under these conditions SM144 selectively labels M^{Pro} in a dose-dependent manner as was observed with recombinant protein. Fluorescence labeling was dose dependently inhibited in the presence of the parent inhibitors, SM141 and SM142 (FIG. 4D), consistent with M^{Pro} being the primary target of these inhibitors. In total, these results confirm the engagement of M^{Pro} by the M^{Pro} inhibitors in the presence of a complex proteome. As a further control, unspiked A549-hACE2 cell lysates were treated with SM144 alone and in the presence of SM141 and SM142. Notably, no proteins were significantly labeled, indicating the high M^{Pro}-selectivity of these inhibitors (FIG. 4E).

The proteome-wide selectivity was evaluated via an orthogonal chemoproteomic assay. A549-hACE2 lysates spiked with recombinant M^{Pro} were treated with SM144 in the presence and absence of a competitive inhibitor, SM141. Subsequently, the labeled proteins were biotinylated with copper-catalyzed click chemistry in the presence of Biotin-azide, and the biotinylated proteins were selectively enriched on Streptavidin-agarose beads, proteolyzed with trypsin and analyzed by chemoproteomic analysis. These studies indicate that M^{Pro} is the most statistically significantly enriched protein, confirming the proteome-wide selectivity of SM144 (FIG. 4F). There were, however, a few cellular off-targets, including glutathione reductase (GSHR), adenosylhomocysteinase (SAHH), aldose reductase (ALDR), S-adenosylmethionine synthase isoform type-2 (METK2), gamma-actin (ACTG), tubulin alpha-4A chain (TBA4A), ubiquitin-like modifier-activating enzyme 1 (UBA1) and junction plakoglobin (PLAK). Notably, all these proteins are negligibly enriched in the presence of SM141, confirming that these proteins are bonafide cellular targets of the M^{Pro} inhibitors (FIG. 4G). However, none of these proteins are related to the entry, replication, and/or exocytosis of SARS-CoV2 in host cells, indicating that they are unlikely to contribute to the antiviral activity of the M^{Pro} inhibitors and may simply be labeled because they are highly abundant proteins with reactive cysteines.

Cathepsins L and B (CatL/B) play important roles by cleaving the Spike protein and thereby facilitating the release of SARS-CoV2 genomic RNA into the cytosol of a host cell. CatL/B are mainly expressed in the lysosome and therefore, cleave the S protein only after the virus is internalized into host cell through endocytosis. Recent studies suggest that the circulating levels of cathepsin L (CatL) are significantly elevated in COVID-19 patients after SARS-CoV2 infection. SARS-CoV2 pseudovirus infection also elevates CatL expression in human cells as well as in hACE2 transgenic mice. Notably, both these cathepsins are cysteine proteases that can be targeted with small molecule inhibitors containing cysteine-reactive warheads. Several reports indicated that the inhibition of either of these Cathepsins, or both, markedly reduces SARS-CoV2 infection. Bogyo and coworkers showed that most of the known M^{Pro} inhibitors also potently inhibit CatL and B. (Padmanabhan, et al. 2020 PLoS Comput. Biol. 16, e1008461; Bosch, et al. 2008 J. Virol. 82, 8887; Gomes, et al. 2020 Front. Cell. Infect. Microbiol. 10, 589505; Pislar, et al. 2020 PLoS Pathog. 16, e1009013; Zhao, et al. 2021 Signal Transduct. Target. Ther. 6, 134; Ashhurst, et al. 2021 J. Med. Chem. 65, 2956-2970; Hashimoto, et al. 2021 Mol. Ther. Nucleic Acids 26, 1107; Liu, et al. 2020 Pharmacol. Ther. 213, 107587; Ma, et al. 2022 ChemMedChem 17, e202100456; Steuten, et al. 2021 ACS Infect. Dis. 7, 1457.)

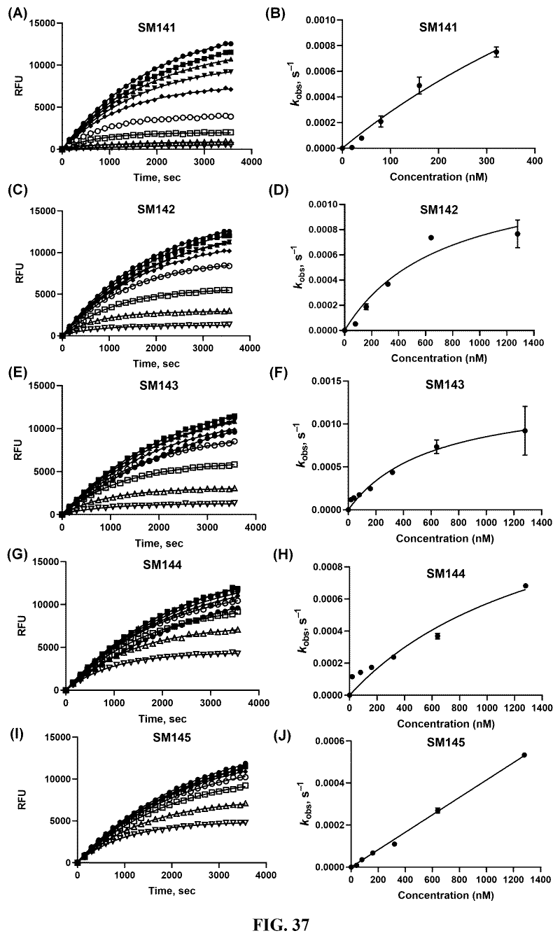

However, no significant enrichment of CatL/B was found in the chemoproteomic analysis (FIGS. 4F and G). Since CatL/B expression is boosted upon SARS-CoV2 infection, it is possible that the M^{Pro} inhibitors target CatL/B once cells are infected. To determine whether the M^{Pro} inhibitors also inhibit CatL/B, commercially available kits were used determined to evaluate the inhibition of CatL/B. Interestingly, SM141-145 are excellent inhibitors of CatL with SM141 being the most potent (Table 1, and FIGS. 36A and 37). The IC₅₀ and k_{inact}/K_{I} values indicate that SM141 is 10-fold more potent for CatL inhibition than M^{Pro}. However, none of these compounds appreciably inhibit CatB (IC₅₀>50 M), indicating that SM141 likely exerts its antiviral activity by selectively targeting both M^{Pro} and CatL (FIG. 36B). Since M^{Pro} acts downstream of the action of CatL, treatment with SM141 can simultaneously block viral replication as well as further entry of the virus into the host cell. SM141 may also inhibit excess CatL that is expressed post-infection, further contributing to its antiviral activity.

Intrigued by the cellular antiviral activity of SM141 and SM142, their efficacy in mouse models of SARS-CoV2 infection was investigated. As such, the pharmacokinetic properties of SM141 and SM142 after intravenous (IV), intraperitoneal (IP) and oral (PO) administration into BALB-6c mice were determined (Table 3). SM141 has more aqueous solubility than SM142. It accumulates in blood plasma in high concentration immediately after dosing (see Cₘₐₓ and Tₘₐₓ), however, this is due to a lower tissue distribution. Most of the administered SM141 circulates in the plasma with a half-life (T_{1/2}) of about an hour. Notably, SM141 has minimal oral bioavailability but did achieve high plasma levels after IP dosing. By contrast, owing to the lower solubility, SM142 required a micelle formulation. SM142 has much higher tissue distribution than SM141 and extensively depots into tissue, resulting in low plasma concentration and longer plasma half-life (see Cₘₐₓ and T_{1/2}).

TABLE 3
Pharmacokinetic parameters of SM141 and SM142

| | SM141 | | SM142 | |
|---|---|---|---|---|
| Parameters | IV | IP | IV | PO |
| T_{1/2} (h) | 0.8 | 0.8 | 2.1 | 2.1 |
| Tₘₐₓ (h) | 0.08 | 0.14 | 0.08 | 0.08 |
| Cₘₐₓ (μM) | 3.9 | 2.7 | 0.4 | 0.6 |
| AUCₗₐₛₜ (μM · h) | 1.3 | 1.6 | 0.4 | 0.5 |
| AUC_{INF}_{_{—}}_{obs} | 42350 | 49620 | 18490 | 20843 |
| (min · ng/mL) | | | | |
| CI_Obs | 71.5 | — | 169 | — |
| (mL/min/kg) | | | | |
| MRT_{INF}_{_{—}}_{obs} (h) | 0.4 | — | 1.9 | — |
| V_{SS}_{_{—}}obs (l/kg) | 1.6 | — | 13.2 | — |
| % Bioavailability | — | 35.2 | — | 37.5 |

IV: Intravenous dosing;
IP: Intraperitoneal dosing;
PO: Oral dosing

Furthermore, by contrast to SM141, SM142 has significant oral absorption. All these pharmacokinetic parameters underscore the possibility of evaluating these two inhibitors in preclinical models of SARS-CoV-2.

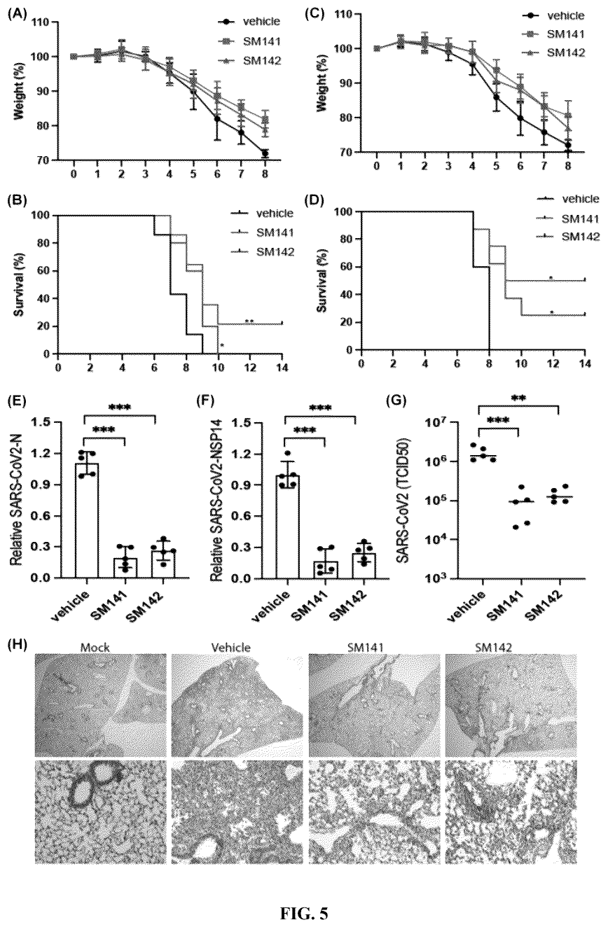

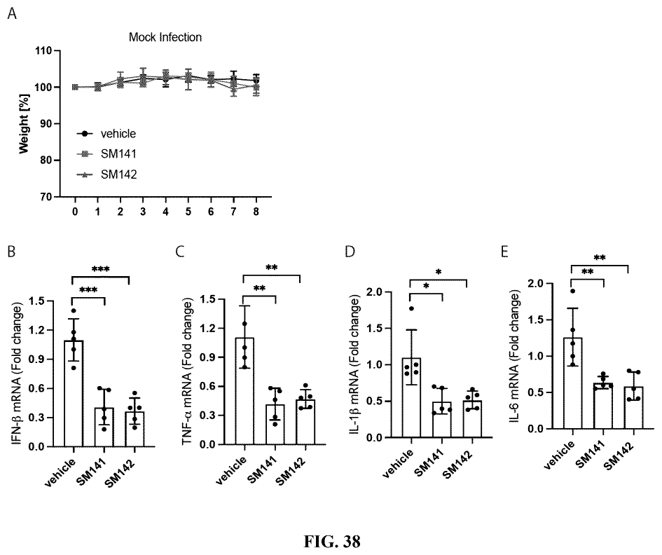

Given the excellent cellular antiviral potency and appreciable pharmacokinetic properties of SM141 and SM142, next investigated was the in vivo antiviral efficacy against SARS-CoV-2 infection. For these studies, K18-hACE2 transgenic mice (K18-ACE2) were used that express human ACE2 under the control of the epithelial cell cytokeratin-18 (K18) promoter as this model provides robust SARS-CoV-2 infection. Notably, both compounds are significantly nontoxic as they cause no significant weight loss in the uninfected mice (FIG. 38A). Intranasal infection of K18-ACE2 mice with SARS-CoV2 resulted in significant weight loss and lethality 7 days post-infection. However, intranasal treatment with a once daily dose of SM141 or SM142 for 3 days starting 2 h prior to infection protects mice from SARS-CoV2-induced weight loss and lethality (FIGS. 5A and B). Furthermore, post-infection administration of SM141 or SM142 via intraperitoneal injection twice daily for 5 days also protects K18-ACE2 mice from SARS-CoV-2-induced weight loss and lethality (FIGS. 5C and D). Notably, the survival of mice is significantly improved in the latter treatment, indicating that these compounds exhibit better antiviral efficacy when administered intraperitoneally. Although SM142 exhibits better pharmacokinetics upon intraperitoneal injection than SM141, SM141 exhibits better antiviral efficacy (FIGS. 5B and D) likely due to the higher potency for M^{Pro} and CatL inhibition. Also investigated was the viral RNA loads and titers of SARS-CoV-2 in the lung tissue after treatment with SM141 and SM142. As shown in FIGS. 5E-G, the viral RNA loads and titers in lung tissues are remarkably lower in the inhibitor-treated mice than in those treated with vehicle control. Reduced inflammatory cytokines and chemokines were detected, including IFN-β, TNF-α, IL-1β and IL-6 in the lung tissues of inhibitor-treated mice consistent with lower viral loads (FIG. 38B-E). In agreement with these results, histopathological analysis of the lung tissues (FIG. 5H) also shows a significant reduction in lung inflammation and improved overall lung pathology upon SM141 or SM142 treatment for 5 days, suggesting that these compounds not only reduce viral loads but also ameliorate SARS-CoV2-triggered inflammatory cytokine storm and lung damage.

### Experimental

### Materials and Methods

L-Phenylalanine, HBTU and HOBt were bought from Chem-Impex International, Inc. N-succinimidyl 3-(propargyloxy)propionate and (2E,4S)-4-(tert-butoxycarbonylamino)-5-[(3′S)-2′-oxo-3′-pyrrolidinyl]-2-pentenoic acid ethyl ester(acrylate warhead) were obtained from Alfa Aesar and Chiramer, LLC., respectively. 4-fluoro-L-phenylalanine, 4-fluoro-D-phenylalanine, D-phenylalanine, DIPEA, anhydrous DMF, anhydrous dichloromethane, triethylamine, trifluoroacetic acid and HPLC-grade acetonitrile were purchased from Sigma-Aldrich. TCEP and streptavidin agarose beads (catalogue no. 20353) were obtained from Thermo Scientific. Precoated silica gel plates were bought from Merck. Cathepsin L (Catalogue no. ab197012) and Cathepsin B (Catalogue no. ab185438) inhibition assay kits were obtained from Abcam. ¹H and ¹³C NMR spectra were recorded in d₆-DMSO as solvent using a Bruker 500 MHz NMR spectrometer. Chemical shift values are cited with respect to SiMe₄(TMS) as the internal standard. Column chromatography was performed using an automated CombiFlash (Biotage) purification system. SM136-145 were purified by reverse-phase HPLC using a semi-preparative C18 column (Agilent, 21.2×250 mm, 10 m) and a water/acetonitrile gradient supplemented with 0.05% trifluoroacetic acid except for Boc-protected inhibitors for which a water/acetonitrile gradient was used. Fluorographs were recorded using a Typhoon scanner with excitation/emission maxima of ˜546/579, respectively. A549-hACE2- and Huh7.5 cells were cultured in Dulbecco's modified Eagle's medium (DMEM) supplemented with 10% (v/v) fetal bovine serum, 100 U/ml penicillin and 100 μg/ml streptomycin. All antiviral study protocols were reviewed and approved by Environmental Health and Safety and Institutional review board at University of Massachusetts Medical School prior to study initiation. All experiments with SARS-CoV-2 were performed in a biosafety level 3 laboratory by personnel equipped with powered air-purifying respirators.

### Synthesis.

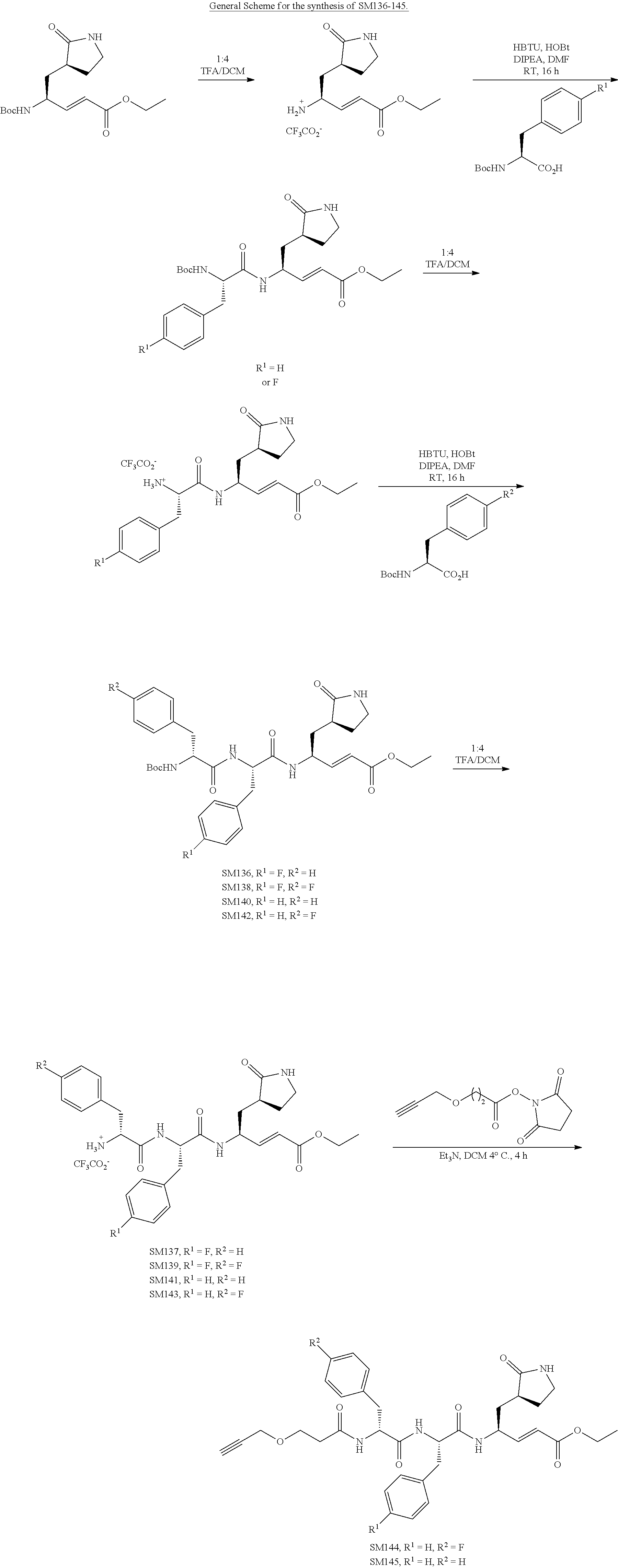

### General Procedure for Amide Coupling

DIPEA (3 eq), HBTU (2 eq) and HOBt (2 eq) were added sequentially to a solution of carboxylic acid (1 eq) and trifluoroacetate salt of amine (1 eq) in anhydrous DMF. The mixture was allowed to stir at room temperature for 12 h under a nitrogen atmosphere. Then the reaction mixture was poured into water and the organics were extracted with dichloromethane (3×). The combined organic extract was washed with water and brine, and dried over anhydrous sodium sulphate. Then the crude mixture was dried in vacuo and was purified by flash column chromatography. The final products that were used in the biochemical and antiviral assays were purified by reverse phase HPLC using a pre-packed C18 column and a water/acetonitrile gradient as the eluent.

### General Procedure for Boc-Deprotection

A Boc-protected compound was dissolved in 1:4 trifluoroacetic acid/dichloromethane (v/v) (10 mL for 1 g of Boc-protected compound) and the mixture was stirred at room temperature for 1 h. Excess trifluoroacetic acid/dichloromethane was evaporated under reduced pressure to afford the free amine as a gummy liquid that was used for subsequent steps without further purification. The trifluoroacetate salts that were used in the biochemical and antiviral assays were purified by reverse phase HPLC using a pre-packed C18 column and a water/acetonitrile (supplemented with 0.05% TFA) gradient as the eluent.

### General Procedure for Installing the N-Terminal Alkyne Handle

A solution of SM141 or SM143 (1 eq) in dichloromethane was treated with triethylamine (3 eq) and N-succinimidyl 3-(propargyloxy)propionate (1.2 eq) at 4° C. for 4 h. Excess solvent was evaporated and the crude mixture was purified by reverse phase HPLC using a pre-packed C18 column and a water/acetonitrile (supplemented with 0.05% TFA) gradient as the eluent.

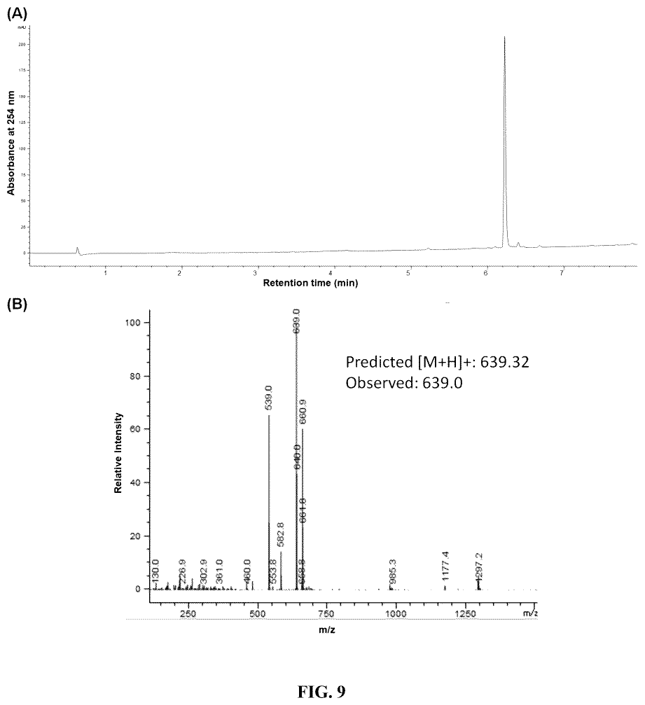

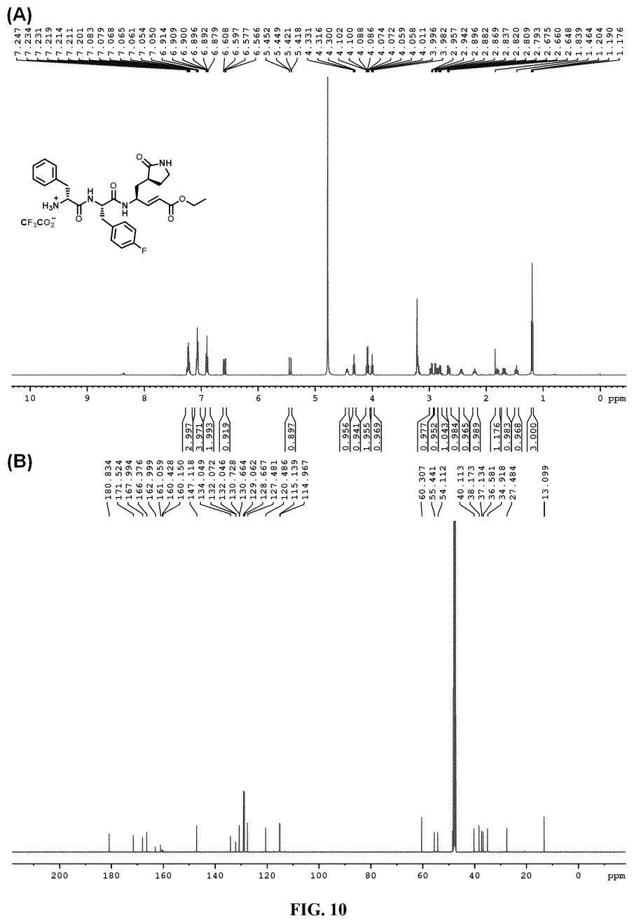

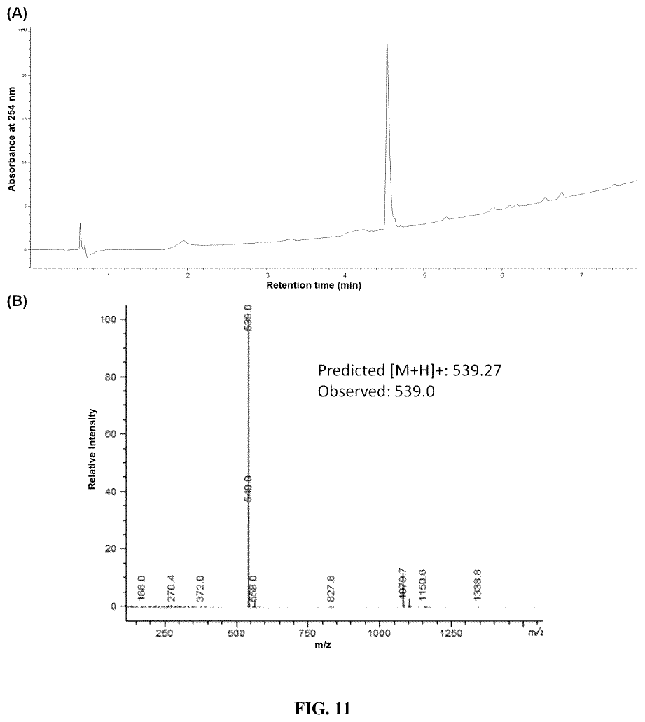

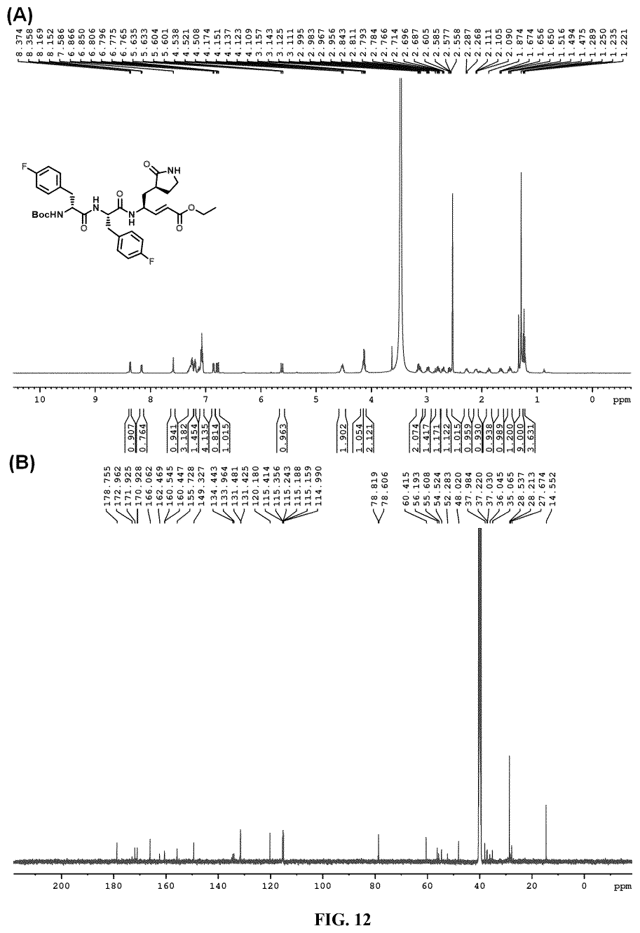

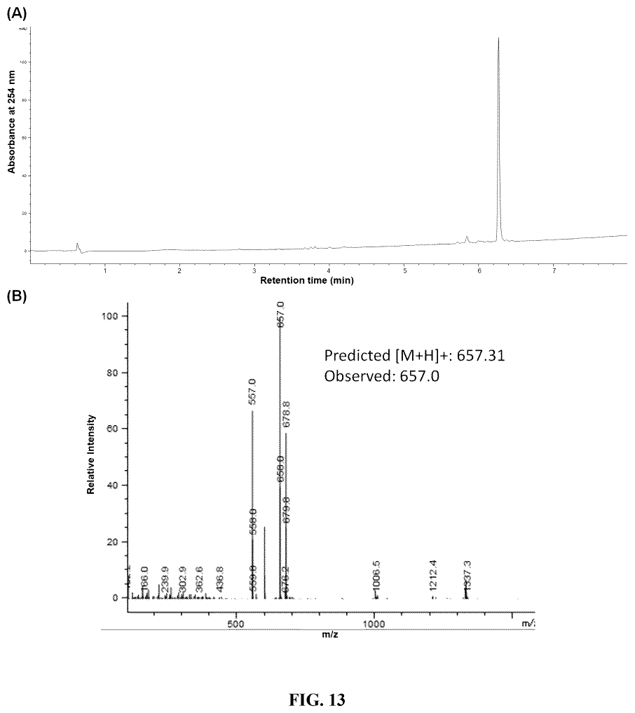

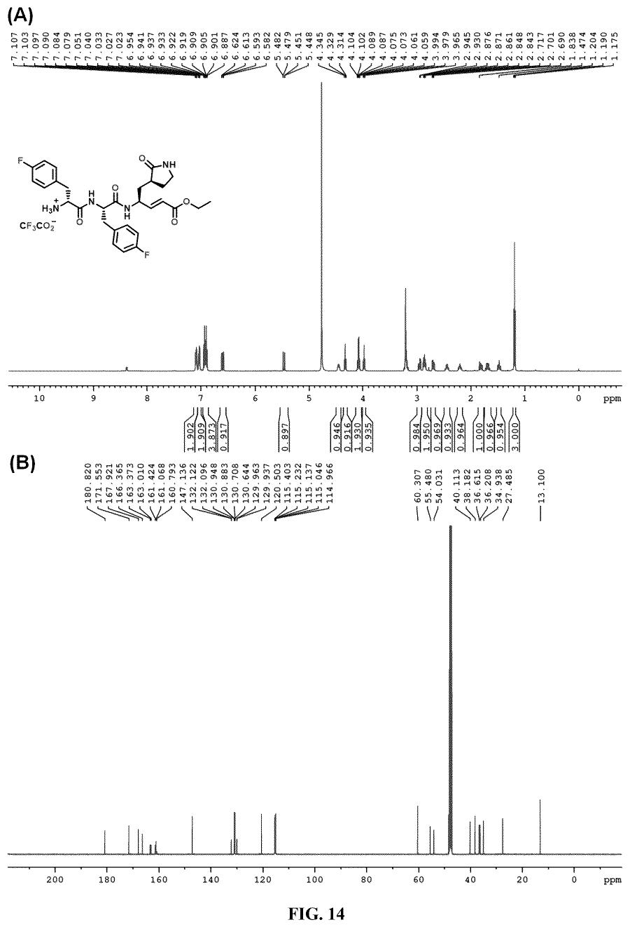

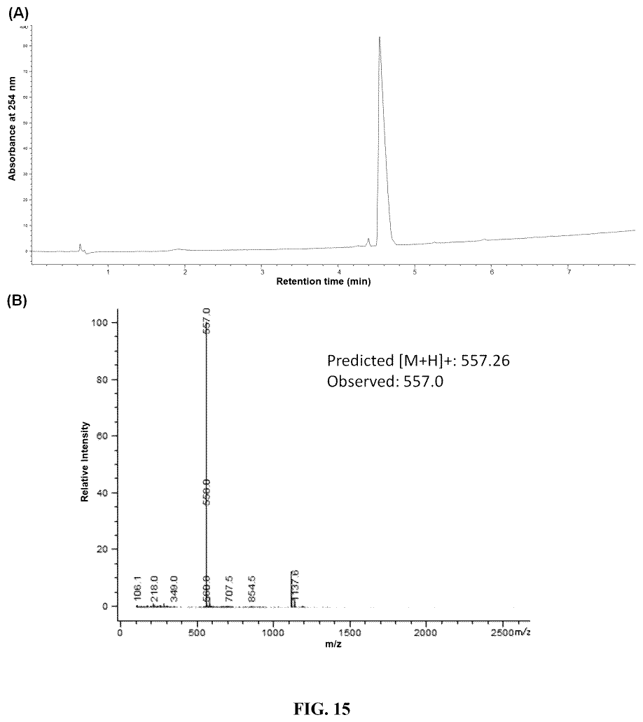

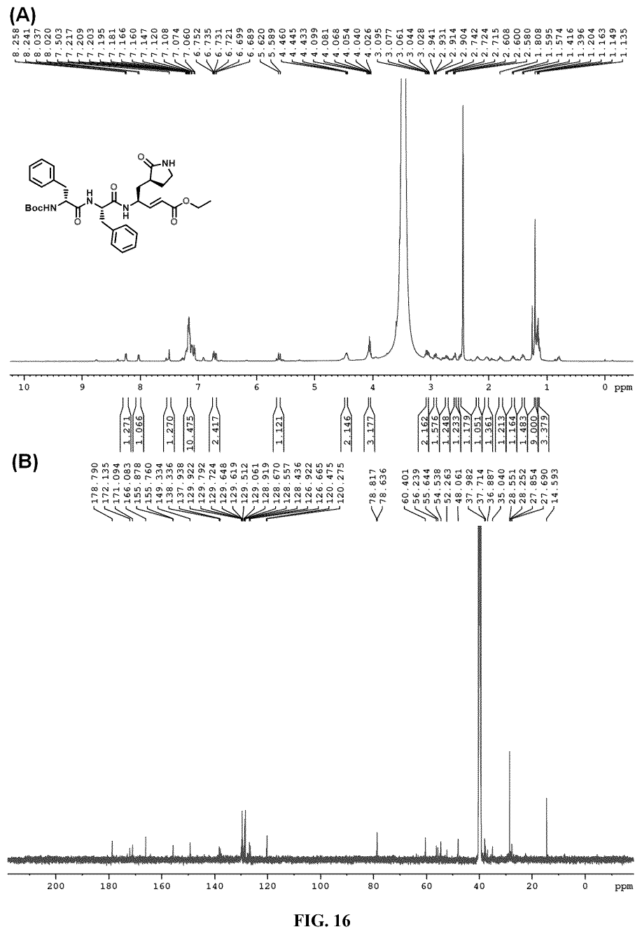

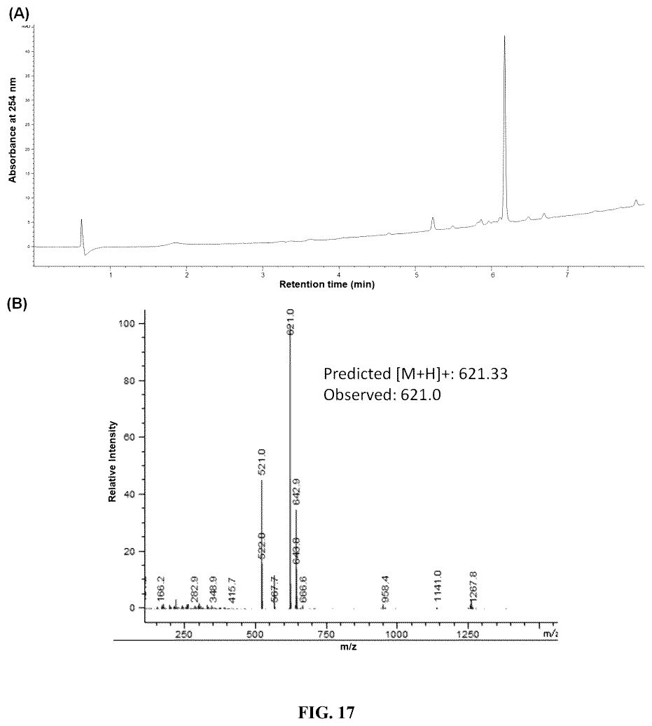

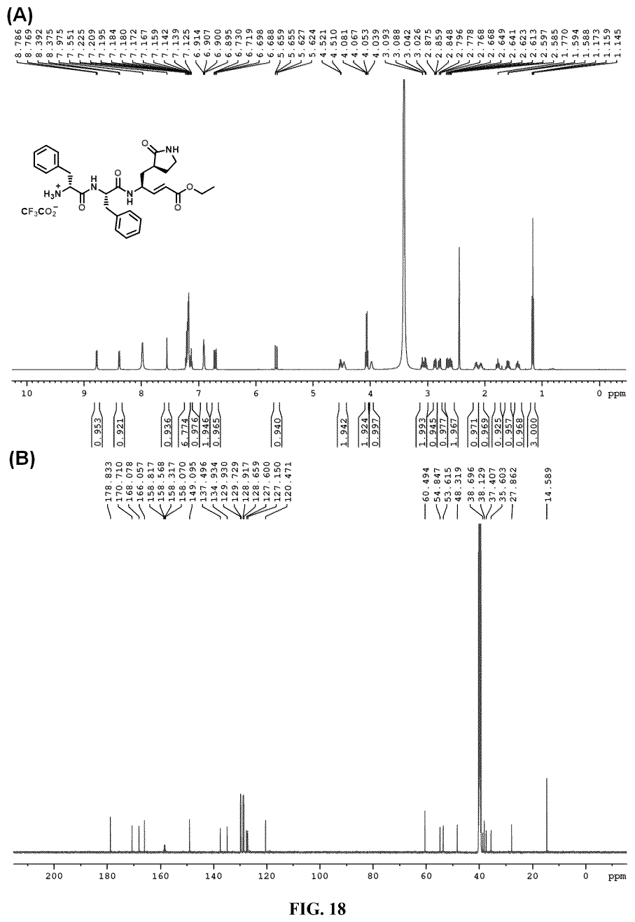

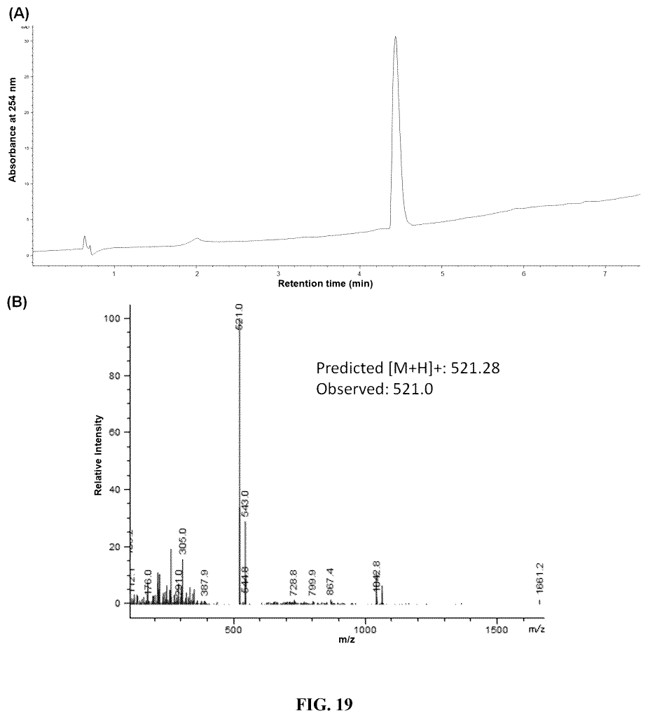

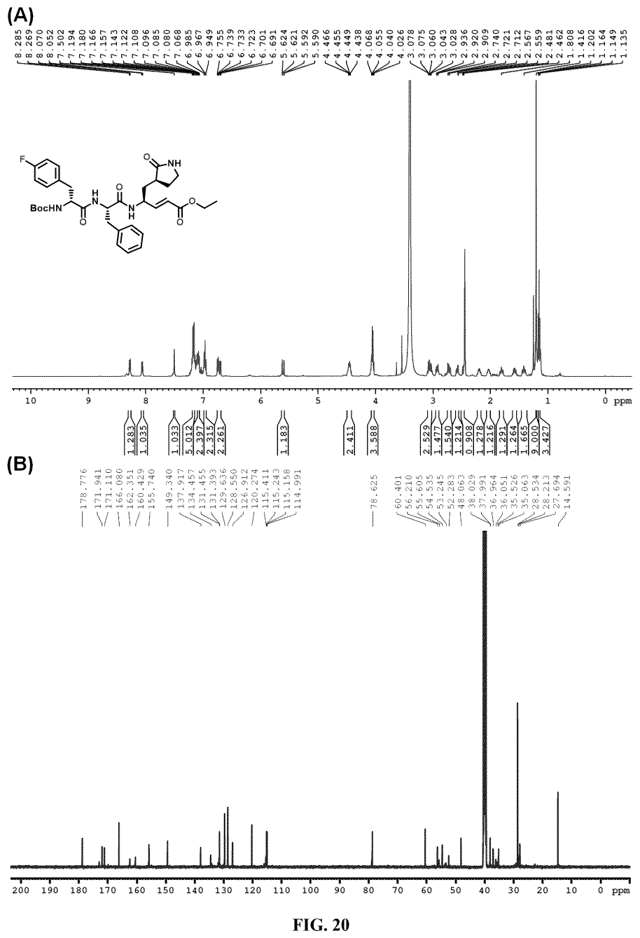

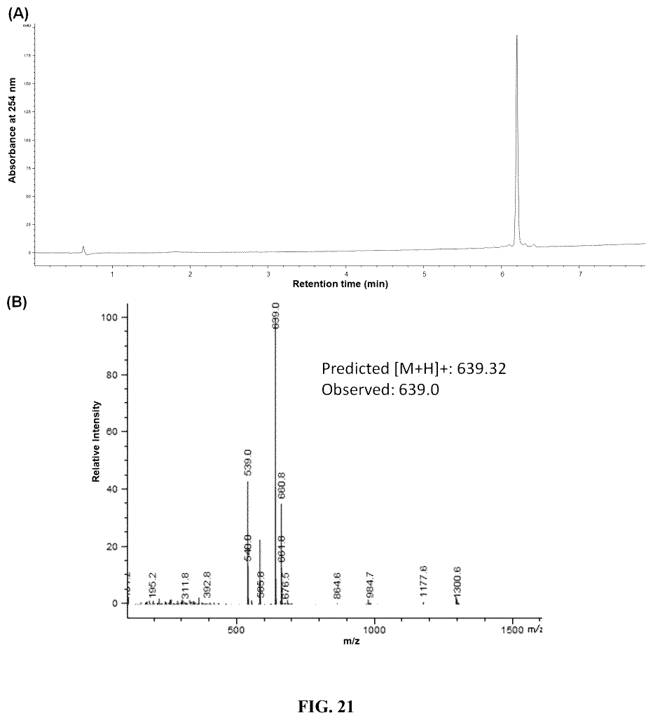

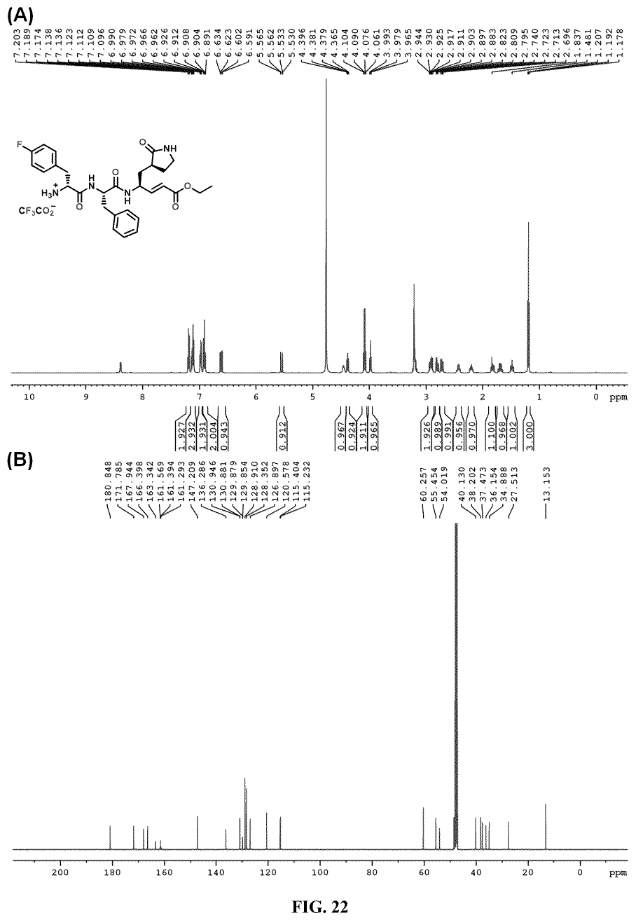

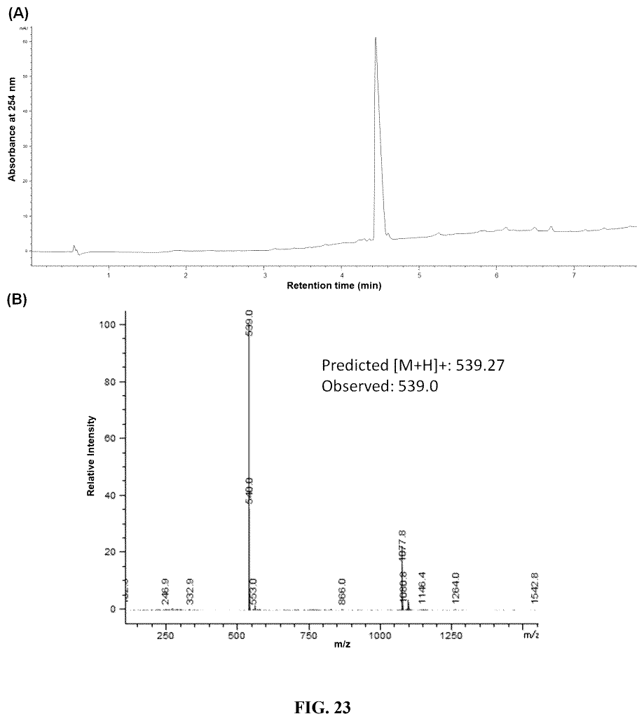

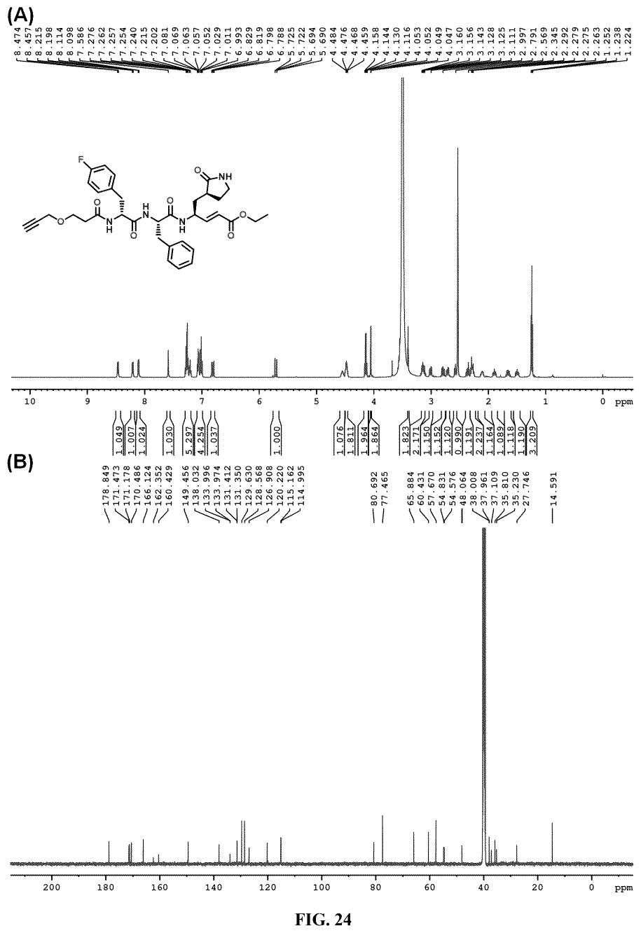

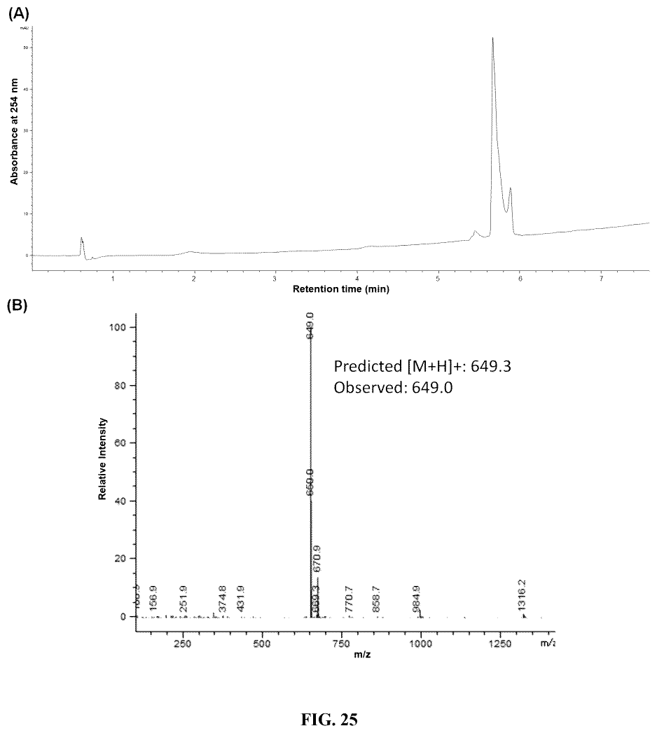

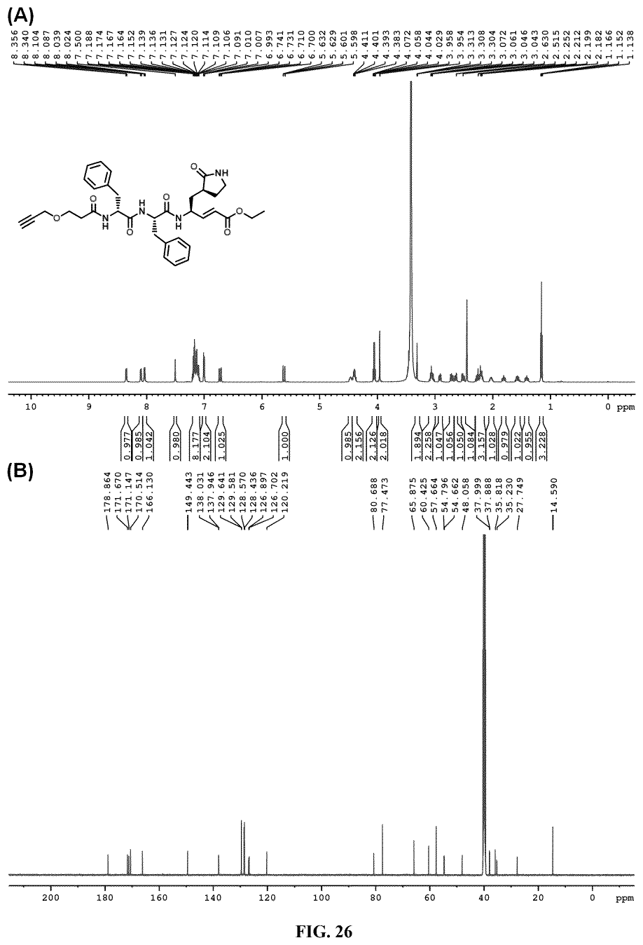

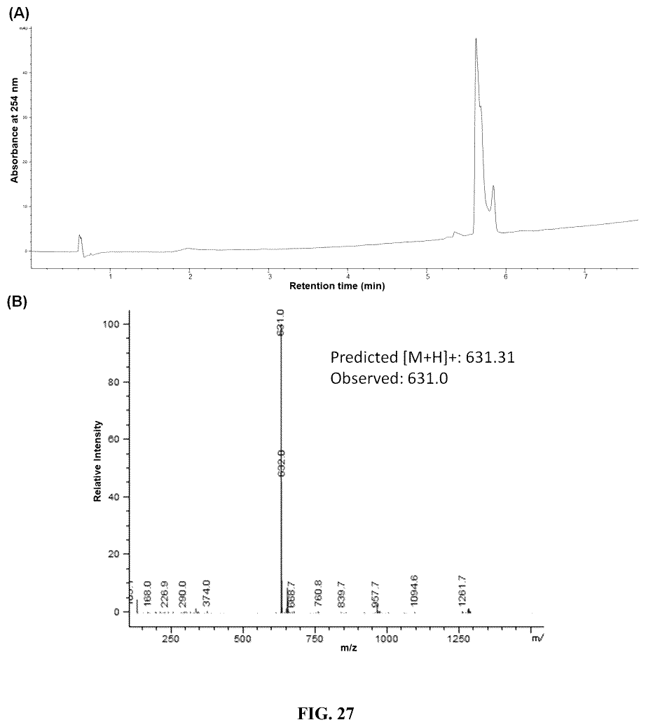

Compounds SM136-145 were thoroughly characterized by ¹H and ¹³C NMR spectroscopy and mass spectrometry. These data are given in FIGS. 7-26. The purity of all these compounds was determined by ¹H NMR spectroscopy and LC-MS analysis. All the tested compounds were >95% pure.

SM136. ¹H NMR (DMSO-d₆) δ (ppm): 8.40 (d, J=8.3 Hz, 1H), 8.19 (d, J=8.7 Hz, 1H), 7.64 (s, 1H), 7.28-7.31 (m, 5H), 7.21 (d, J=7.6 Hz, 2H), 7.11 (t, J=8.8 Hz, 2H), 6.89 (d, J=7.9 Hz, 1H), 6.81-6.85 (dd, 1H), 5.65-5.68 (m, 1H), 4.53-4.60 (m, 2H), 4.20-4.24 (m, 1H), 4.15-4.19 (m, 2H), 3.13-3.22 (m, 2H), 2.99-3.03 (m, 1H), 2.82-2.88 (m, 1H), 2.75-2.78 (m, 1H), 2.63-2.68 (m, 1H), 2.30-2.36 (m, 1H), 2.13-2.18 (m, 1H), 1.90-1.95 (m, 1H), 1.66-1.74 (m, 1H), 1.51-1.57 (m, 1H), 1.34 (s, 9H), 1.28 (t, J=7.1 Hz, 3H); ¹³C NMR (DMSO-d₆) δ (ppm): 178.8, 173.1, 172.1, 170.9, 166.1, 162.5, 160.5, 155.9, 155.7, 149.3, 138.3, 138.0, 134.0, 131.5, 131.4, 129.6, 129.5, 128.7, 128.4, 126.9, 126.7, 120.2, 115.4, 115.2, 78.8, 78.6, 60.4, 56.2, 55.6, 54.6, 52.3, 48.0, 38.0, 37.8, 37.1, 36.9, 35.0, 28.6, 28.2, 27.7, 14.6; ESI-MS (m/z) calculated for C₃₄H₄₃N₄O₇F₁ [M+H]⁺: 639.32, found 639.0.

SM137. ¹H NMR (DMSO-d₆) δ (ppm): 7.20-7.21 (m, 3H), 7.05-7.08 (m, 4H), 6.87-6.91 (m, 2H), 6.57-6.61 (dd, 1H), 5.42-5.45 (m, 1H), 4.43-4.45 (m, 1H), 4.32 (t, J=7.8 Hz, 1H), 4.06-4.10 (m, 2H), 4.00 (t, J=7.3 Hz, 1H), 2.94-2.98 (m, 1H), 2.85-2.90 (m, 1H), 2.79-2.84 (m, 1H), 2.63-2.68 (m, 1H), 2.42-2.45 (m, 1H), 2.19-2.20 (m, 1H), 1.78-1.84 (m, 1H), 1.64-1.72 (m, 1H), 1.44-1.49 (m, 1H), 1.19 (t, J=7.1 Hz, 3H); ¹³C NMR (DMSO-d₆) δ (ppm): 180.8, 171.5, 168.0, 166.4, 163.0, 161.1, 160.4, 160.2, 147.1, 134.0, 132.1, 132.0, 130.7, 130.6, 129.1, 128.7, 127.5, 120.5, 115.1, 115.0, 60.3, 55.4, 54.1, 40.1, 38.2, 37.1, 36.6, 34.9, 27.5, 13.1; ESI-MS (m/z) calculated for C₂₉H₃₅N₄O₅F [M+H]⁺: 539.27, found 539.0.

SM138. ¹H NMR (DMSO-d₆) δ (ppm): 8.37 (d, J=8.3 Hz, 1H), 8.16 (d, J=8.7 Hz, 1H), 7.58 (s, 1H), 7.23-7.31 (m, 3H), 7.18-7.21 (m, 1H), 7.05-7.09 (m, 4H), 6.86 (d, J=8.0 Hz, 1H), 6.76-6.81 (dd, 1H), 5.60-5.64 (dd, 1H), 4.49-4.56 (m, 2H), 4.11-4.17 (m, 3H), 3.09-3.18 (m, 2H), 2.96-3.03 (m, 1H), 2.77-2.87 (m, 1H), 2.66-2.72 (m, 1H), 2.56-2.60 (m, 1H), 2.25-2.31 (m, 1H), 2.08-2.13 (m, 1H), 1.85-1.90 (m, 1H), 1.61-1.69 (m, 1H), 1.47-1.52 (m, 1H), 1.29 (s, 9H), 1.24 (t, J=7.1 Hz, 3H); ¹³C NMR (DMSO-d₆) δ (ppm): 178.8, 173.0, 171.9, 170.9, 166.1, 162.5, 160.5, 160.4, 155.7, 149.3, 134.4, 134.0, 131.5, 131.4, 120.2, 115.4, 115.3, 115.2, 115.2, 115.1, 115.0, 78.8, 78.6, 60.4, 56.2, 55.6, 54.5, 52.3, 48.0, 38.0, 37.2, 37.0, 36.0, 35.1, 28.5, 28.2, 27.7, 14.6; ESI-MS (m/z) calculated for C₃₄H₄₂N₄O₇F₂ [M+H]⁺: 657.31, found 657.0.

SM139. ¹H NMR (DMSO-d₆) δ (ppm): 7.08-7.10 (m, 2H), 7.02-7.05 (m, 2H), 6.89-6.95 (m, 4H), 6.58-6.62 (dd, 1H), 5.45-5.48 (m, 1H), 4.43-4.47 (m, 1H), 4.33 (t, J=7.8 Hz, 1H), 4.06-4.10 (m, 2H), 3.98 (t, J=7.2 Hz, 1H), 2.93-2.97 (m, 1H), 2.83-2.89 (m, 2H), 2.67-2.72 (m, 1H), 2.41-2.48 (m, 1H), 2.18-2.23 (m, 1H), 1.79-1.84 (m, 1H), 1.65-1.73 (m, 1H), 1.45-1.50 (m, 1H), 1.19 (t, J=7.1 Hz, 3H); ¹³C NMR (DMSO-d₆) δ (ppm): 180.8, 171.6, 167.9, 166.4, 163.4, 163.0, 161.4, 161.1, 160.8, 147.1, 132.1, 132.0, 130.9, 130.8, 130.7, 130.6, 130.0, 129.9, 120.5, 115.4, 115.2, 115.1, 115.0, 114.9, 60.3, 55.5, 54.0, 40.1, 38.2, 36.6, 36.2, 34.9, 27.5, 13.1; ESI-MS (m/z) calculated for C₂₉H₃₄N₄O₅F₂ [M+H]: 557.26, found 557.0.

SM140. ¹H NMR (DMSO-d₆) δ (ppm): 8.25 (d, J=8.3 Hz, 1H), 8.03 (d, J=8.6 Hz, 1H), 7.50 (s, 1H), 7.15-7.27 (m, 10H), 6.69-6.75 (m, 2H), 5.56-5.66 (dd, 1H), 4.41-4.46 (m, 2H), 4.03-4.10 (m, 3H), 3.03-3.10 (m, 2H), 2.90-2.94 (m, 1H), 2.70-2.80 (m, 1H), 2.58-2.63 (m, 1H), 2.48-2.51 (m, 1H), 2.17-2.21 (m, 1H), 2.00-2.06 (m, 1H), 1.91-1.97 (m, 1H), 1.78-1.84 (m, 1H), 1.55-1.61 (m, 1H), 1.40-1.44 (m, 1H), 1.20 (s, 9H), 1.15 (t, J=7.0 Hz, 3H); ¹³C NMR (DMSO-d₆) δ (ppm): 178.8, 172.1, 171.1, 166.1, 155.9, 149.3, 138.3, 137.9, 129.9, 129.8, 129.7, 129.6, 129.6, 129.5, 129.1, 128.9, 128.7, 128.6, 128.4, 126.9, 126.7, 120.5, 120.3, 78.8, 78.6, 60.4, 56.2, 55.6, 54.5, 52.3, 48.1, 38.0, 37.7, 36.9, 35.0, 28.6, 28.3, 27.9, 27.7, 14.6; ESI-MS (m/z) calculated for C₃₄H₄₄N₄O₇ [M+H]⁺: 621.33, found 621.0.

SM141. ¹H NMR (DMSO-d₆) δ (ppm): 8.78 (m, J=8.2 Hz, 1H), 8.38 (d, J=8.5 Hz, 1H), 7.60 (s, 1H), 7.16-7.21 (m, 6H), 7.11-7.14 (m, 1H), 6.90-6.91 (m, 2H), 6.69-6.73 (dd, 1H), 5.62-5.66 (dd, 1H), 4.42-4.54 (m, 2H), 4.02-4.08 (m, 2H), 3.97 (s, 1H), 3.01-3.07 (m, 2H), 2.85-2.89 (dd, 1H), 2.77-2.81 (dd, 1H), 2.57-2.67 (m, 2H), 2.12-2.18 (m, 1H), 2.04-2.09 (m, 1H), 1.74-1.80 (m, 1H), 1.55-1.63 (m, 1H), 1.39-1.45 (m, 1H), 1.16 (t, J=7.1 Hz, 3H); ¹³C NMR (DMSO-d₆) δ (ppm): 178.8, 170.7, 168.1, 166.1, 158.8, 158.6, 158.3, 158.1, 149.1, 137.5, 134.9, 129.9, 129.7, 128.9, 128.7, 127.6, 127.1, 120.5, 60.5, 54.8, 53.6, 48.3, 38.7, 38.1, 37.4, 35.6, 27.9, 14.6; ESI-MS (m/z) calculated for C₂₉H₃₆N₄O₅ [M+H]⁺: 521.28, found 521.0.

SM142. ¹H NMR (DMSO-d₆) δ (ppm): 8.28 (d, J=8.4 Hz, 1H), 8.06 (d, J=8.6 Hz, 1H), 7.50 (s, 1H), 7.14-7.19 (m, 5H), 7.07-7.12 (m, 2H), 6.97 (t, J=8.8 Hz, 1H), 6.69-6.75 (m, 2H), 5.59-5.62 (dd, 1H), 4.42-4.48 (m, 2H), 4.02-4.08 (m, 3H), 3.01-3.09 (m, 2H), 2.90-2.95 (m, 1H), 2.69-2.74 (m, 1H), 2.56-2.59 (m, 1H), 2.48 (s, 1H), 2.16-2.23 (m, 1H), 2.00-2.05 (m, 1H), 1.78-1.84 (m, 1H), 1.56-1.61 (m, 1H), 1.38-1.44 (m, 1H), 1.20 (s, 9H), 1.15 (t, J=7.1 Hz, 3H); ¹³C NMR (DMSO-d₆) δ (ppm): 178.8, 171.9, 171.1, 166.1, 162.4, 160.4, 155.7, 149.3, 137.9, 134.5, 131.5, 131.4, 129.6, 128.5, 126.9, 120.3, 115.4, 115.2, 115.1, 115.0, 78.6, 60.4, 56.2, 55.6, 54.5, 53.2, 52.3, 48.1, 38.0, 37.9, 37.0, 36.1, 35.5, 35.1, 28.5, 28.2, 27.7, 14.6; ESI-MS (m/z) calculated for C₃₄H₄₃N₄O₇F₁ [M+H]⁺: 639.32, found 639.0.

SM143. ¹H NMR (DMSO-d₆) δ (ppm): 7.17-7.20 (m, 2H), 7.10-7.14 (m, 2H), 6.96-6.99 (m, 2H), 6.89-6.93 (m, 2H), 6.59-6.63 (dd, 1H), 5.53-5.57 (dd, 1H), 4.43-4.47 (m, 1H), 4.36-4.40 (m, 1H), 4.06-4.10 (m, 2H), 3.98 (t, J=7.0 Hz, 2H), 2.89-2.94 (m, 2H), 2.77-2.82 (m, 1H), 2.70-2.74 (m, 1H), 2.39-2.46 (m, 1H), 2.17-2.23 (m, 1H), 1.79-1.85 (m, 1H), 1.65-1.73 (m, 1H), 1.45-1.51 (m, 1H), 1.19 (t, J=7.2 Hz, 3H); ¹³C NMR (DMSO-d₆) δ (ppm): 180.8, 171.8, 167.9, 166.4, 163.3, 161.6, 161.4, 161.3, 147.2, 136.3, 130.9, 130.8, 129.9, 129.8, 128.9, 128.4, 126.9, 120.6, 115.4, 115.2, 60.3, 55.4, 54.0, 40.1, 38.2, 37.5, 36.2, 34.9, 27.5, 13.2; ESI-MS (m/z) calculated for C₂₉H₃₅N₄O₅F₁ [M+H]f: 539.27, found 539.0.

SM144. ¹H NMR (DMSO-d₆) δ (ppm): 8.30 (d, J=8.3 Hz, 1H), 8.21 (d, J=8.7 Hz, 1H), 8.10 (d, J=7.7 Hz, 1H), 7.59 (s, 1H), 7.18-7.29 (m, 5H), 6.99-7.08 (m, 4H), 6.79-6.83 (dd, 1H), 5.69-5.73 (dd, 1H), 4.51-4.57 (m, 1H), 4.45-4.50 (m, 2H), 4.12-4.16 (m, 2H), 4.04-4.05 (dd, 2H), 3.40 (t, J=2.4 Hz, 2H), 3.11-3.17 (m, 2H), 2.99-3.02 (m, 1H), 2.76-2.81 (m, 1H), 2.69-2.73 (m, 1H), 2.56-2.59 (m, 1H), 2.33-2.39 (m, 1H), 2.25-2.30 (m, 2H), 2.07-2.13 (m, 1H), 1.86-1.92 (m, 1H), 1.63-1.69 (m, 1H), 1.47-1.52 (m, 1H), 1.24 (t, J=7.1 Hz, 3H); ¹³C NMR (DMSO-d₆) δ (ppm): 178.8, 171.5, 171.2, 170.5, 166.1, 162.4, 160.4, 149.5, 138.0, 134.0, 133.9, 131.4, 131.3, 129.6, 128.6, 126.9, 120.2, 115.2, 115.0, 80.7, 77.5, 65.9, 60.4, 57.7, 54.8, 54.6, 48.1, 38.0, 37.9, 37.1, 35.8, 35.2, 27.7, 14.6; ESI-MS (m/z) calculated for C₃₅H₄₁N₄O₇F₁ [M+H]⁺: 649.30, found 649.0.

SM145. ¹H NMR (DMSO-d₆) δ (ppm): 8.35 (d, J=8.3 Hz, 1H), 8.10 (d, J=8.7 Hz, 1H), 8.03 (d, J=7.7 Hz, 1H), 7.50 (s, 1H), 7.08-7.20 (m, 8H), 6.99-7.01 (dd, 2H), 6.70-6.74 (dd, 1H), 5.60-5.63 (dd, 1H), 4.44-4.49 (m, 1H), 4.37-4.43 (m, 2H), 4.03-4.07 (m, 2H), 3.96 (d, J=2.4 Hz, 2H), 3.31 (t, J=2.4 Hz, 2H), 3.01-3.09 (m, 2H), 2.89-2.93 (m, 1H), 2.68-2.73 (m, 1H), 2.62-2.66 (m, 1H), 2.48-2.53 (m, 1H), 2.16-2.29 (m, 3H), 1.99-2.05 (m, 1H), 1.78-1.84 (m, 1H), 1.53-1.61 (m, 1H), 1.38-1.44 (m, 1H), 1.15 (t, J=7.1 Hz, 3H); ¹³C NMR (DMSO-d₆) δ (ppm): 178.9, 171.7, 171.1, 170.5, 166.1, 149.4, 138.0, 137.9, 129.6, 129.5, 128.6, 128.4, 126.9, 126.7, 120.2, 80.7, 77.5, 65.9, 60.4, 57.7, 54.8, 54.7, 48.1, 38.0, 37.9, 35.8, 35.2, 27.7, 14.6; ESI-MS (m/z) calculated for C₃₅H₄₂N₄O₇ [M+H]⁺: 631.31, found 631.0.

### Expression and Purification of SARS-CoV-2 M^{Pro}

SARS2-Mpro was expressed and purified as previously described (PMID: 33503819). Briefly, the SARS-CoV-2-M^{Pro} plasmid was transformed into Escherichia coli strain HI-Control™ BL21(DE3) (Lucigen, Middleton, WI, USA). The transformed cells were pre-cultured at 37° C. in LB medium with ampicillin (100 g/mL) overnight, and the cell culture was inoculated into TB medium containing 50 mM sodium phosphate (pH 7.0) and ampicillin (100 μg/mL). When the OD600 value reached ˜2.0, 0.5 mM IPTG was added to induce SARS-CoV-2-M^{Pro} expression and the cell culture was further incubated overnight at 20° C. Cells were harvested by centrifugation at 5000 rpm for 20 min, resuspended in lysis buffer (50 mM Tris-HCl (pH 8.0), 400 mM NaCl, 1 mM TCEP) and lysed by a cell disruptor. The lysate was clarified by ultracentrifugation at 18,000 rpm for 50 min. The supernatant was loaded onto a HisTrap FF column (Cytiva, Marlborough, MA, USA) equilibrated with lysis buffer, washed with lysis buffer and followed by elution using elution buffer (50 mMTris-HCl pH 8.0, 400 mM NaCl, 500 mM imidazole, 1 mM TCEP) with a linear gradient of imidazole ranging from 0 to 500 mM. The fractions of M^{Pro}-His tag were mixed with GST-PreScission protease-His-tag at a molar ratio of 5:1 to remove the C-terminal His tag. The PreScission protease treated M^{Pro} was applied to nickel column to remove the GST-PreScission protease-His-tag and protein with uncleaved His-tag. His-tag cleaved M^{Pro} in the flow-through was further purified by size-exclusion chromatography (HiLoad™ 16/60 Superdex 75 [Cytiva, Marlborough, MA, USA]) and stored in 20 mM HEPES pH 7.5, 150 mM NaCl, 1 mM TCEP.

### Expression and Purification of SARS-CoV-2 PL^{Pro}

PL^{Pro} was expressed and purified as reported earlier.¹ Briefly, the pET28a(+)-SARS-CoV-2 PLpro construct was transformed into E. coli Artic express (DE3) (Agilent). The transformed cells were pre-cultured at 37° C. in LB medium with Kanamycin (30 g/mL) overnight, and the cell culture was inoculated into LB medium containing Kanamycin (30 μg/mL) at a starting OD600 of 0.05. When OD₆₀₀ value reached ˜0.8-1.0, the cell culture was incubated for 1 hour in an incubator equilibrated at 4° C. with agitation at 180 rpm prior to induction. Proteins were induced by 0.5 mM IPTG and 50 mL of a 1M potassium phosphate buffer pH7.4 (KH₂PO₄ K21HPO₄), followed by additional incubation overnight at 18° C. Cells were harvested by centrifugation at 5000 rpm for 20 min, resuspended in lysis buffer (50 mMTris-HCl (pH 8.0), 500 mM NaCl, 20 mM Imidazole, 5% glycerol and 1 mM TCEP) and lysed by a cell disruptor. The lysate was clarified by ultracentrifugation at 18000 rpm for 50 min. The supernatant was loaded onto a HisTrap FF column (GE Healthcare) equilibrated with lysis buffer, washed with lysis buffer and followed by elution using elution buffer (50 mMTris-HCl (pH 8.0), 500 mM NaCl, 500 mM imidazole, 5% glycerol and 1 mM TCEP) with a linear gradient of imidazole ranging from 20 mM to 500 mM.

Determination of IC₅₀ values for SARS-CoV-2 M^{Pro}

Inhibition assays were performed by monitoring the cleavage of a fluorogenic M^{Pro} substrate (Dabcyl-KTSAVLQSGFRKM-E(Edans)-NH₂) by M^{Pro} in the presence of the inhibitors. Cleavage of this substrate at the C-terminal end of glutamine (Q) by M^{Pro} produces fluorescence. A M^{Pro} solution (12.5 nM final) in the assay buffer (50 mM HEPES pH 7.5, 150 mM NaCl, 1 mM EDTA, 1 mM DTT) was treated with either test compound (0-80 M for D-FF(R/Cit)-(CK/CMKyne); 0-1.25 mM for SM136-145) or 2 μL assay buffer with 50% DMSO (2% final DMSO) for 20 min. Then the peptide-substrate was added (30 μM final) and the reaction mixture was incubated for 30 min at 25° C. The total volume of the assay mixture was 50 μL. End-point analysis was performed by recording the fluorescence at absorption/emission wavelength of 340/485 nm using an EnVision 2105 plate reader. The IC₅₀ values were determined from the Hill slope fits using GraphPad Prism. All measurements were done in triplicate.

Determination of K_{I} values for SARS-CoV-2 M^{Pro}

A solution of M^{Pro} (12.5 nM final) in the assay buffer (50 mM HEPES pH 7.5, 150 mMNaCl, 1 mM EDTA, 1 mM DTT) was treated with the MPro peptide-substrate (30 μM final) and test inhibitor (0-80 M for D-FF(R/Cit)-(CK/CMKyne); 0-1.25 mM for SM136-145) simultaneously. The total volume of the assay mixture was 50 μL. The initial velocity of enzyme-catalyzed peptide cleavage was determined by monitoring the fluorescence emission of the product over a period of 5-10 min. Fluorescence was measured at absorption/emission wavelength of 340/485 nm using an EnVision 2105 plate reader. The K_{I} values for competitive inhibition of the test compounds were determined from the nonlinear least squares fits of kinetic data to equation 1,

$$\frac{{v}_{i}}{{v}_{0}} = \frac{[ s ] + {K}_{m}}{[ S ] + {K}_{m} ( 1 + \frac{[ I ]}{{K}_{i}} )} \qquad ( 1 )$$

where νₒ is the initial velocity of uninhibited reaction and νᵢ is the initial velocity in the presence of inhibitor. All measurements were done in triplicate.
Determination of k_{inact}/K_{I} values for SARS-CoV-2 M^{Pro}

Time- and concentration-dependent inactivation assays were done by following a similar protocol as described for Kᵢ determination. M^{Pro} (12.5 nM final) was treated with the peptide-substrate (30 μM final) and test inhibitor (0-80 μM for D-FF(R/Cit)-(CMK/CMKyne); 0-1.25 mM for SM136-145) at time=0 to initiate inactivation process. The total volume of the assay mixture was 50 μL. Time-dependent increase in fluorescence was monitored at 485 nm with excitation at 340 nm for a period of 20-25 min. The observed rates (k_{obs}) for the peptide cleavage in both the presence and absence of inhibitor were determined by fitting to a single-phase association equation. The resulting k_{obs} values were plotted against the concentrations of inhibitor and linear fits were used to derive the slope that afforded the k_{inact}/K_{I} values. All measurements were done in triplicate.

Determination of IC₅₀ values for SARS-CoV-2 PL^{Pro}

PL^{Pro} inhibition assays were performed using a fluorogenic substrate (Z-RLRGG-AMC) that produces fluorescence upon cleavage by PL^{Pro}. A PL^{Pro} solution (30 nM final) in the assay buffer (50 mM HEPES, pH 7.5, 1 mM DTT) was treated with either test compound (0-80 μM for D-FF(R/Cit)-(CMK/CMKyne); 0-1.25 mM for SM136-145) or 2 μL assay buffer with 50% DMSO (2% final DMSO) for 20 min. Then the peptide-substrate was added (20 μM final) and the reaction mixture was incubated for 30 min at 25° C. The total volume of the assay mixture was 50 μL. End-point analysis was performed by recording the fluorescence at absorption/emission wavelength of 340/460 nm using an EnVision 2105 plate reader. The IC₅₀ values were determined from the Hill slope fits using GraphPad Prism. All measurements were done in triplicate.

Determination of IC₅₀ and k_{inact}/K_{I} values for Cathepsins L and B

IC₅₀ and k_{inact}/K_{I} values of the inhibitors were determined using commercially available inhibition assay kits (Abcam) by following the manufacturer's protocol. For IC₅₀ values, enzymes were pretreated with test inhibitor (0-50 μM) for 20 min prior to adding the fluorogenic substrate, and end-point fluorescence was recorded after 30 min. For the k_{inact}/K_{I} values, enzymes were treated with inhibitor and substrate simultaneously and increase in fluorescence was monitored over 60 min. The observed rates (k_{obs}) were determined by fitting to a single-phase association equation. The resulting k_{obs} values were plotted against the concentrations of inhibitor and the data were fit to equation 2 to derive the k_{inact}/K_{I} values,

$${k}_{obs} = {k}_{inact} [ I ] / ( {K}_{I} + [ I ] ) , \qquad ( 2 )$$

using GraphPad Prism, where k_{inact} is the maximal rate of inactivation, K_{I} is the concentration of inhibitor that affords half-maximal inactivation, and [I] is the concentration of inhibitor. When k_{obs} with [I] varied linearly, k_{inact}/K_{I} was determined from the slope of the line. All the experiments were performed in triplicate. All fluorescence measurements were taken at absorption/emission wavelength of 400/505 nm using an EnVision 2105 plate reader.
Crystallization of M^{Pro} in Complex with Inhibitors

Most of the SARS-CoV-2 M^{Pro} crystals (7MAW, 7MAX, 7MAZ, 7MB0, 7MB1, 7MB2 and 7MB3) were generated as previously described.² Briefly, the condition reliably producing crystals was discovered using the MCSG-1 crystal screen (Anatrace), Well B2, containing 25% (w/v) PEG 3350 and 0.1 M bis-tris-methane pH 5.5 and 0.2 M sodium chloride. The SARS2-M^{Pro}-inhibitor complexes were grown at room temperature by hanging drop vapor diffusion method in a 24-well VDX hanging-drop tray (Hampton Research). The crystals grew between 13-20% (w/v) PEG 3350 with a protease concentration of 5-8 mg/mL with 10-fold molar excess of peptide (3.0-4.5% DMSO). Conditions were mixed with the precipitant solution at a 1:1 ratio (1 μL:1 μL or 2 μL:2 μL) and micro-seeded (1:1-1:10 dilution) with a cat whisker. Crystals appeared overnight and grew to diffraction quality within a week. As data was collected at 100 K, cryogenic conditions consisted of the precipitant solution supplemented with 25% glycerol.

Three of the complexes (7MAT, 7MAU and 7MAV) required an alternate crystallization condition. A condition that produced crystals with an alternate morphology was discovered using the MCSG-2 crystal screen (Anatrace), Well E4, containing 1.6 M Ammonium Sulfate, 0.1 M MES:NaOH pH 6.5, and 10% (v/v) Dioxane. The SARS2-M^{Pro}-inhibitor complexes were grown at room temperature by hanging drop vapor diffusion method in a 24-well VDX hanging-drop tray (Hampton Research). After optimization, the crystals grew best between 1.6-1.8 M Ammonium Sulfate and 0.1 M MES pH 6.5 with 3% Dioxane and a protease concentration of 6 mg/mL with 3-fold molar excess of inhibitor (5% DMSO). Conditions were mixed with the precipitant solution at a 1:1 ratio (1 μL:1 μL) and micro-seeded (1:1-1:2 dilution) with a cat whisker. Crystals appeared overnight and grew to diffraction quality within a week. As data was collected at 100 K, cryogenic conditions consisted of the precipitant solution supplemented with 25% glycerol.

### X-Ray Diffraction Data Collection and Structure Determination

D-FFR-CMK and D-FFR-CMKyne were flash frozen under a cryostream when mounting the crystals at in-house Rigaku_Saturn944 X-ray system (Rigaku, The Woodlands, TX, USA). All other complexes were flash frozen in liquid nitrogen and shot at the Brookhaven National Laboratory NSLS-II Beamline 17-ID-2 (FMX). The cocrystal diffraction intensities from the Rigaku system were indexed, integrated, and scaled using HKL3000.³ The structures shot at the APS were indexed, integrated, and scaled using XDS.⁴ All structures were solved using molecular replacement with PHASER.⁵, Model building and refinement were performed using Coot and Phenix.^{6,7} During refinement, optimized stereochemical weights were utilized. Ligands were designed in Maestro and the output sdf files were used in the Phenix program eLBOW⁸ to generate cif files containing atomic positions and constraints necessary for ligand refinement. Iterative rounds of crystallographic refinement were carried out until convergence was achieved. To limit bias throughout the refinement process, five percent of the data were reserved for the free R-value calculation.⁹ MolProbity¹⁰ was applied to evaluate the final structure before deposition in the PDB.^{11,12} Structure analysis, superposition and figure generation was done using PyMOL. X-ray data collection and crystallographic refinement statistics are presented in Table 4.

Dose-Dependent Labeling of Recombinant M^{Pro} with SM144

M^{Pro} (2 μM final) was added to assay mixture (1×PBS, 1 mM DTT) containing various concentrations of SM144 (or equal volume of DMSO for control) and the mixture was incubated at 25° C. for 30 min. TAMRA-N₃ (50 μM), TCEP (1 mM), TBTA (0.3 mM) and freshly prepared copper (II) sulphate (1 mM) were sequentially added to the reaction mixture and the tubes were gently tumbled at room temperature for 2 h. Then the reactions were quenched with 5×SDS-PAGE loading dye, heated at 95° C. for 10 min and loaded onto 4-15% gradient SDS-PAGE gel. Protein bands were visualized by recording in-gel fluorescence using a typhoon scanner (excitation/emission maxima ˜546/579 nm). All the experiments were performed in duplicate.

Limit of Detection for Labeling of Recombinant M^{Pro} with SM144

Various concentrations of M^{Pro} (0-80 μmol) were added to assay mixtures (1×PBS, 1 mM DTT) containing SM144 (5 μM final). The reaction mixture was incubated at 25° C. for 30 min followed by sequential addition of TAMRA-N₃ (50 μM), TCEP (1 mM), TBTA (0.3 mM) and freshly prepared copper (II) sulphate (1 mM). The tubes were then gently tumbled at 25° C. for 2 h. Reactions were quenched with 5×SDS-PAGE loading dye, heated at 95° C. for 15 min and loaded onto 4-15% gradient SDS-PAGE gel. Protein bands were visualized by recording in-gel fluorescence using a typhoon scanner (excitation/emission maxima ˜546/579 nm). All the experiments were performed in duplicate.

### Target Engagement Assay—Labeling of Recombinant M^{Pro} by SM144 in the Presence of SM141

M^{Pro} (2 μM final) was added to assay mixtures (1×PBS, 1 mM DTT) containing various concentrations of SM141 (0-50 μM) and SM144 (5 μM final). The reaction mixture was incubated at 25° C. for 30 min followed by sequential addition of TAMRA-N₃ (50 μM), TCEP (1 mM), TBTA (0.3 mM) and freshly prepared copper (II) sulphate (1 mM). The tubes were then gently tumbled at 25° C. for 2 h. Reactions were quenched with 5×SDS-PAGE loading dye, heated at 95° C. for 15 min and loaded onto 4-15% gradient SDS-PAGE gel. Protein bands were visualized by recording in-gel fluorescence using a typhoon scanner (excitation/emission maxima ˜546/579 nm). All the experiments were performed in duplicate.

Labelling of A549-hACE2Lysate (with or without Spiked M^{Pro}) with SM144 in the Presence and Absence of SM141 or SM142

A549-hACE2 cells were grown in the DMEM medium (supplemented with 10% heat-inactivated fetal bovine serum, 100 units/mL penicillin and 100 μg/mL streptomycin). Upon reaching ˜90% confluence, cells were collected using a cell scraper, harvested by centrifugation at 3,000 rpm for 5 min and were resuspended in 1×PBS supplemented with 1% NP-40. Then the cells were lysed using a probe sonicator and lysates were cleared by centrifugation at 21000 g for 15 min. Soluble proteins in the lysate were quantified by DC-assay (Bio-Rad).

A549-hACE2 lysate (2 mg/mL, 50 μL total) spiked with or without recombinant M^{Pro} (250 nM final) was treated with DTT (1 mM final) and various concentrations of SM144 (0-5 μM final). For competition experiments, DTT (1 mM final), SM144 (2.5 μM final) and various concentrations of SM141 or SM142 (2.5-25 μM final) were added to A549-hACE2 lysate (2 mg/mL, 50 μL total) followed by the addition of recombinant M^{Pro} (250 nM final) or equal volume of PBS (for control). All these reactions were incubated at 25° C. for 30 min. Then TAMRA-N₃ (100 μM), TCEP (1 mM), TBTA (0.3 mM) and freshly prepared copper (II) sulphate (4 mM) were added sequentially to the reaction mixture. The tubes were then gently tumbled at room temperature for 2 h. The precipitated proteins were collected by centrifugation at 5000 rpm for 6 min. The protein pellets were resuspended in 5×SDS-PAGE loading dye, heated at 95° C. for 10 min and separated by SDS-PAGE. Protein bands were visualized by recording in-gel fluorescence using a typhoon scanner (excitation/emission maxima ˜546/579 nm). All the experiments were performed in duplicate.

Enrichment of Proteins Labelled by SM144 (in the Presence and Absence of SM141) in A549-hACE2 Lysate Spiked with M^{Pro} on Streptavidin-Agarose

A549-hACE2 cell lysate was prepared as described earlier. Endogenous biotinylated proteins were precleared by incubating the lysates with streptavidin-agarose beads (25 μL for 1 mg of total protein in the lysate) at room temperature for 1 h with constant mixing on an end-over-end shaker. The mixture was centrifuged (1200 g, 3 minute) to separate the beads and the supernatant (pre-cleared lysate). Pre-cleared lysates (2 mg/mL, 1 mL final) were treated with DTT (1 mM final), recombinant M^{Pro} (250 nM final) and SM144 (1 μM final) in the presence and absence of SM141 (25 μM final). The assay mixture was incubated at 25° C. for 30 min. Then Biotin-N₃ (200 μM), TCEP (1 mM), TBTA (0.3 mM) and freshly prepared CuSO₄ (4 mM) were added sequentially, and the tubes were gently tumbled at room temperature for 2 h. The precipitated proteins were collected by centrifugation at 5,000 rpm for 10 min. The protein pellets were washed with ice-cold acetone and resuspended in 1.2% SDS in PBS. The solutions were then diluted with PBS to a final SDS concentration of 0.2% and were incubated with streptavidin-agarose beads (200 μL for 2 mg of total protein) at 4° C. for 16 h on an end-over-end shaker. Next, the mixtures were incubated at 25° C. for 3 h. Then the streptavidin beads were collected by centrifugation at 1200 g for 3 min and were washed with 2 M urea (1×5 mL), 0.2% SDS in PBS (2×5 mL), PBS (3×5 mL) and water (3×5 mL). The beads were collected by centrifugation at 1200 g for 3 min between washes. The washed beads were resuspended in 6M urea (500 μL) in 100 mM triethylammonium bicarbonate (TEAB, pH 8.5) and the bound proteins were reduced with dithiothreitol (10 mM) at 65° C. for 20 min. Then the reduced thiols were alkylated by treating with iodoacetamide (20 mM final) at 37° C. in dark for 30 min. The beads were pelleted by centrifugation at 1200 g for 3 min and were treated with a premixed solution of 2M urea in 100 mM TEAB (200 μL), 100 mM CaCl₂ (2 μL) and trypsin (4 μL of 20 μg reconstituted in 40 μL of 100 mM TEAB) at 37° C. for 12 h. The digested peptides were separated from the beads by centrifugation and the beads were washed twice with 100 mM TEAB (50 μL). DMSO-, SM144- and SM144/SM141-treated peptide digests were treated with 4 μL of 20% HCHO (light formaldehyde), 4 μL of 20% D¹²CDO (medium formaldehyde) and 4 μL of 20% D¹³CDO (heavy formaldehyde), respectively. 20 μL of 0.6 M sodium cyanoborohydride (for medium- and light formaldehyde labeled samples) and 20 μL of 0.6 M sodium cyanoborodeuteride (for heavy formaldehyde labeled samples) were then added to the samples, and the samples were incubated at room temperature for 2 h. The samples were cooled on ice and the reactions were quenched with 4 μL of 20% ammonium hydroxide. 8 μL of formic acid was then added to the samples. Light-, medium- and heavy formaldehyde labeled samples were mixed together and the mixtures were then desalted using a Pierce C18 spin column (catalogue no 89870) according to the manufacturer' protocol. The dried peptide pellets were stored at −20° C. for proteomic analysis. All the experiments were performed in triplicate.

### Proteomic Analysis and Data Processing

Data was acquired using a NanoAcquity UPLC (Waters Corporation, Milford, MA) coupled to an Orbitrap Fusion LumosTribrid (Thermo Fisher Scientific, Waltham, MA) mass spectrometer. Peptides were trapped and separated using an in-house 100 m I.D. fused-silica pre-column (Kasil frit) packed with 2 cm ProntoSil (Bischoff Chromatography, DE) C18 AQ (200 Å, 5 μm) media and configured to an in-house packed 75 μm I.D. fused-silica analytical column (gravity-pulled tip) packed with 25 cm Magic (Bruker, Billerica, MA) C18AQ (100 Å, 3 μm) media, respectively. Peptides were eluted with mobile phase A (0.1% formic acid in water) and mobile phase B (0.1% formic acid in acetonitrile). 3.8 μL sample was injected, and the peptides were trapped at a flow rate of 4 μL/min with 5% mobile phase B for 4 min, followed by a gradient elution with 5-35% B at a flow rate of 300 nL/min over 120 min (total run time 145 min). Mass spectra were acquired over m/z 375-1500 Da with a resolution of 120,000 (m/z 200). Tandem mass spectra were acquired using data-dependent acquisition (3 sec cycle) with an isolation width of 1.6 Da, HCD collision energy of 30%, resolution of 15,000 (m/z 200), maximum injection time of 50 ms, and an AGC target of 50,000.

Raw data was processed and searched using Maxquant 1.6.14 and its integrated Andromeda search engine using the Swiss-Prot human and SARS-CoV-2 database. Search parameters were as follows: tryptic digestion with up to 2 missed cleavages; peptide N-terminal acetylation, methionine oxidation, N-terminal glutamine to pyroglutamate conversion were specified as variable modifications. The monoisotopic mass increment of 28.0313, 32.0564 and 36.0757 Da for the light-, medium- and heavy dimethyl labels, respectively were set as variable modification on the peptide N-termini and lysine residues. Carbamidomethylation of cysteines was set as static modification. Protein grouping, dimethyl ratio calculations and downstream statistics were done in Scaffold Q+S 4.8.9 (Proteome Software, Portland, OR).

### Cellular Antiviral Assay

A549-hACE2 cells or Huh7.5 cells cultured in Dulbecco's modified Eagle's medium supplemented with 10% (v/v) fetal bovine serum, 100 U/ml penicillin and 100 g/ml streptomycin were seeded on 24-well plates; after 16 h were incubated with SARS-CoV-2 or OC43 at a MOI=0.05 for 1 h. The media was then removed and replaced with fresh media containing the indicated doses of inhibitors, the inhibitors were maintained in the media throughout the experiment. After 24 h infection, the cells were collected with Trizol (Invitrogen) and total RNA was extracted with the Direct-zol RNA miniprep kit (Zymo) per the manufacturer's instructions. Equal amounts of RNA were reverse-transcribed using the iScript cDNA synthesis kit (Bio-Rad). Diluted cDNAs (1:100 final) were subjected to qPCR analysis using iQ SYBR Green Supermix reagent (Bio-Rad). Gene expression levels of SARS-CoV-2-N mRNA were normalized to GAPDH a common housekeeping gene. Relative mRNA expression was calculated by the change-in-cycling-threshold method as 2-ddC(t). The specificity of amplification was assessed for each sample by melting curve analysis. IC₉₀ and IC₅₀ was determined by GraphPad Prism 8 software.

### Cytotoxicity Assay

A549-hACE2 cells were treated with indicated doses of inhibitors for 24 h, the media were collected for lactate dehydrogenase (LDH) assay using the CytoTox 96@Non-Radioactive Cytotoxicity Assay kit (Promega). Supernatant samples are transferred to a 96-well plate and an equal volume of CytoTox 96® Reagent is added to each well and incubated for 30 min. Stop Solution is added, and the absorbance is measured at 490 nm using a plate reader.

### Antiviral Activity in the SARS-CoV-2-Infected Mice

All animal experiments were approved by the Institutional Animal Care Use Committees at the University of Massachusetts Chan Medical School. Animal were kept in a specific pathogen free (SPF) environment. Hemizygous K18-hACE2 C57BL/6J mice (strain: 2B6.Cg-Tg (K18-ACE2)2Prlmn/J) were obtained from The Jackson Laboratory. 8-12-week-old male and female mice were anesthetized with isoflurane and infected intranasally with 3×10⁴ PFU of SARS-CoV-2. The mice were treated with SM141 or SM142 intranasally (10 mg/kg, once 2 h prior to the infection and two more doses on the consecutive days after infection) or intraperitoneally treatment with 25 mg/kg SM141 or SM142 twice daily for 5 days. Mice were monitored daily for weight loss and survival. Lung samples were collected at indicated time points and placed in a bead homogenizer tube with 1 ml of DMEM+2% FBS for homogenization, then 100 μl of this mixture was placed in PBS for tittering or in 300 μl Trizol LS (Invitrogen) for RNA extraction. For histology, lungs were perfused with 10 U/mL Heparin, intratracheally inflated with 10% buffered-formalin and dissected from mice. Tissues were fixed in 4% paraformaldehyde overnight and embedded in 10% paraffin. Five micrometer thin sections were stained by H&E. Histomorphology, grading of histology scores and evaluation of inflammation of each H&E slide was evaluated by Applied Pathology Systems.

### Sample Preparations for Evaluating the Pharmacokinetic Parameters

Pharmacokinetics were evaluated in male C57Bl/6 mice, approximately 25 g. Test compounds were formulated at 0.3 mg/mL for intravenous dosing and 1 mg/mL for oral gavage. Formulation concentrations reflect free-base concentrations. Formulations were prepared by dissolving in DMSO (20× stock) and mixing 1:1 with tween-80. Prior to dosing, the formulation was diluted with sterile saline. The final formulation was 5% DMSO, 5% tween-80, 90% saline (v:v:v). Mice (n=3) were dosed at 10 μL per gram body weight. Each mouse had blood collected nine times (5, 15, 30, 60, 120, 240, 360, 480, and 1440 minutes) using heparin coated hematocrit tubes and a tail nick. A total of 25 μL blood was collected at each time point. The hematocrit tubes were sealed with a wax plug and held on ice until plasma could be isolated via centrifugation in a microcentrifuge fit with a hematocrit rotor.

Drug levels were determined from 5 μL plasma. Plasma was treated with 75 μL 90/10 acetonitrile/water containing Carbamazepine as an internal standard (IS). Samples were loaded to a 96-well Millipore Multiscreen Solvinter 0.45 micron low binding PTFE hydrophilic filter plate and the filtrate was directly analyzed. All plasma samples were treated with 75 μL 90/10 acetonitrile/water with Carbamazepine as internal standard (IS) to extract the analyte and precipitate protein. The plates were agitated on ice for approximately five minutes prior to centrifugation into a collection plate. Separate standard curves were prepared in blank mouse plasma and brain homogenate and processed in parallel with the samples. The filtrate was directly analyzed by LC-MS/MS. HPLC and MS/MS parameters are provided in Table 4.

TABLE 4
X-ray data collection and crystallographic refinement statistics

| | D-FFR- | D-FFR- | D-FFCit- | | | | | | |
|---|---|---|---|---|---|---|---|---|---|
| Inhibitor | CMK | CMKyne | CMKyne | SM137 | SM139 | SM141 | SM143 | SM144 | SM145 |
| Protein | M^{Pro} WT | M^{Pro} WT | M^{Pro} WT | M^{Pro} WT | M^{Pro} WT | M^{Pro} WT | M^{Pro} WT | M^{Pro} WT | M^{Pro} WT |
| PDB ID | 7MAT | 7MAU | 7MAV | 7MAX | 7MAZ | 7MB0 | 7MB1 | 7MB2 | 7MB3 |
| Data | | | | | | | | | |
| Collection | | | | | | | | | |
| Location | UMass | UMass | NSLS-II | NSLS-II | NSLS-II | NSLS-II | NSLS-II | NSLS-II | NSLS-II |
| Wavelength | 1.54178 | 1.54178 | 0.979356 | 0.979356 | 0.979356 | 0.979356 | 0.979356 | 0.979356 | 0.979356 |
| (Å) | | | | | | | | | |
| Space group | P12₁1 | P12₁1 | P12₁1 | P12₁1 | P12₁1 | P12₁1 | P12₁1 | P12₁1 | P1 |
| a, b, c (Å) | 48.0. | 49.3, | 50.0, | 48.2, | 47.8, | 47.7, | 47.9, | 47.6, | 65.5, |
| | 105.8, | 106.6, | 106.5, | 106.0, | 105.3, | 104.5, | 104.9, | 104.5, | 67.3, |
| | 54.4 | 54.0 | 53.6 | 54.2 | 52.9 | 52.2 | 52.5 | 52.5 | 117.9 |
| α, β, γ (°) | 90, | 90, | 90, | 90, | 90, | 90, | 90, | 90, | 92.0, |
| | 104.0, | 104.1, | 104.6, | 104.0, | 104.1, | 104.2, | 104.1, | 104.1, | 90.3, |
| | 90 | 90 | 90 | 90 | 90 | 90 | 90 | 90 | 119.0 |
| Resolution | 23.6- | 23.2- | 48.4- | 29.4- | 29.0- | 46.2- | 29.5- | 46.2- | 29.4- |
| (Å) | 2.74 | 1.95 | 1.91 | 1.98 | 1.70 | 1.54 | 1.43 | 1.89 | 1.81 |
| | (2.84- | (2.02- | (1.98- | (2.06- | (1.76- | (1.60- | (1.48- | (1.96- | (1.88- |
| | 2.74) | 1.95) | 1.91) | 1.98) | 1.70) | 1.54) | 1.43) | 1.89) | 1.81) |
| Total | 50785 | 141577 | 202190 | 72274 | 104561 | 579610 | 170908 | 170001 | 549566 |
| Reflections | (4637) | (11507) | (20189) | (6864) | (7691) | (55622) | (11484) | (16675) | (51080) |
| Unique | 13621 | 38475 | 40919 | 36477 | 53454 | 72666 | 86824 | 39442 | 154181 |
| Reflections | (1321) | (3624) | (4045) | (3511) | (4337) | (7085) | (6158) | (3850) | (14215) |
| Redundancy | 3.7 | 3.7 | 4.9 | 2.0 | 2.0 | 8.0 | 2.0 | 4.3 | 3.6 |
| | (3.5) | (3.2) | (5.0) | (2.0) | (1.8) | (7.9) | (1.9) | (4.3) | (3.6) |
| Completeness | 98.2 | 97.8 | 97.2 | 99.4 | 95.9 | 99.7 | 94.0 | 99.5 | 96.4 |
| (%) | (95.1) | (92.9) | (96.1) | (96.0) | (77.8) | (97.9) | (63.8) | (97.9) | (88.9) |
| Average I/σ | 16.9 | 17.5 | 8.3 | 13.7 | 10.2 | 11.4 | 11.0 | 8.0 | 10.2 |
| | (3.0) | (4.6) | (2.4) | (1.8) | (1.8) | (3.4) | (1.8) | (2.4) | (1.8) |
| R_{merge}^{a} | 0.061 | 0.050 | 0.110 | 0.026 | 0.044 | 0.121 | 0.033 | 0.110 | 0.069 |
| | (0.425) | (0.257) | (0.603) | (0.350) | (0.344) | (0.981) | (0.341) | (0.543) | (0.603) |
| Rₚᵢₘ | 0.037 | 0.030 | 0.055 | 0.026 | 0.044 | 0.046 | 0.033 | 0.059 | 0.042 |
| | (0.264) | (0.168) | (0.294) | (0.350) | (0.344) | (0.373) | (0.341) | (0.289) | (0.367) |
| CC ½ | 0.998 | 0.996 | 0.993 | 0.999 | 0.997 | 0.996 | 0.998 | 0.993 | 0.997 |
| | (0.870) | (0.921) | (0.867) | (0.783) | (0.730) | (0.822) | (0.784) | (0.798) | (0.868) |
| Refinement | | | | | | | | | |
| Mol per A.U. | 2 | 2 | 2 | 2 | 2 | 2 | 2 | 2 | 6 |
| R_{factor} (%)^{c} | 19.5 | 17.1 | 20.1 | 20.1 | 16.4 | 17.9 | 18.8 | 16.7 | 19.5 |
| R_{free} (%)^{d} | 25.9 | 21.5 | 24.5 | 24.3 | 20.6 | 20.0 | 21.4 | 21.1 | 23.3 |
| RMSD^{b} in: | | | | | | | | | |
| Bond lengths | 0.003 | 0.011 | 0.010 | 0.005 | 0.018 | 0.009 | 0.010 | 0.016 | 0.005 |
| (Å) | | | | | | | | | |
| Bond angles (°) | 0.570 | 0.860 | 1.140 | 0.710 | 1.400 | 0.950 | 1.080 | 1.240 | 0.720 |
| Ramachandrans: | | | | | | | | | |
| Favored (%) | 96.69 | 98.34 | 96.53 | 97.52 | 98.35 | 97.67 | 98.01 | 97.35 | 97.23 |
| Allowed (%) | 2.81 | 1.50 | 3.47 | 2.15 | 1.32 | 2.16 | 1.99 | 2.48 | 2.38 |
| Outliers (%) | 0.05 | 0.17 | 0.00 | 0.33 | 0.33 | 0.17 | 0.00 | 0.17 | 0.39 |
| B-factors: | | | | | | | | | |
| Average | 50.4 | 27.7 | 41.1 | 48.2 | 25.2 | 24.3 | 23.0 | 30.9 | 37.3 |
| Macromolecules | 50.4 | 26.7 | 40.9 | 48.1 | 24.5 | 23.5 | 22.1 | 30.5 | 37.0 |
| Ligands | 52.9 | 45.3 | 49.1 | 54.3 | 24.0 | 24.1 | 21.6 | 32.8 | 45.8 |
| Solvent | 38.4 | 34.0 | 42.3 | 47.8 | 33.1 | 33.0 | 31.3 | 36.8 | 40.1 |

Values in parentheses are for the highest resolution shell.
^{a}R_{sym} = Σ | I − <I>|/Σ I, where I = observed intensity, <I> = average intensity over symmetry equivalent
^{b}RMSD, root mean square deviation.
^{c}R_{factor} = Σ || Fₒ| − |F_{c}||/Σ|Fₒ|.
^{d}R_{free} was calculated from 5% of reflections, chosen randomly, which were omitted from the refinement process.

TABLE 5
LCMS Settings for the evaluation of pharmacokinetics parameters
LC(Shimadzu UFLC XR) conditions

| Column | Thermo Betasil C18 5μ, 50 × 2.1 mm |
|---|---|
| Mobile phase | A: Water with 0.1% Formic Acid |
| | B: Acetonitrile with 0.1% Formic Acid |
| Flow rate (ml/min) | 0.35 |
| Temperature (° C.) | 35 |
| Injection volume(μl) | 10 |

Gradient elution conditions:

| | Mobile | Mobile |
|---|---|---|
| Time (min) | phase A (%) | phase B (%) |
| 0.2 | 90 | 10 |
| 0.5 | 90 | 10 |
| 2.0 | 5 | 95 |
| 3.0 | 5 | 95 |
| 4.0 | 90 | 10 |
| 5.9 | 90 | 10 |

MS(API5500) conditions

| | | I.S. | | I.S. |
|---|---|---|---|---|
| Compound | SM141 | (Carbamazepine) | SM142 | (Carbamazepine) |
| MRM(+) | 522.3/210.1 | 237.2/194.1 | 639.1/539.1 | 237.2/194.1 |
| Collision Gas | 7 | | 7 | |
| Curtain GAS | 35 | | 35 | |
| Ion Source Gas1 | 55 | | 55 | |
| Ion Source Gas2 | 50 | | 50 | |
| Ion Spray Voltage | 5500 | | 5500 | |
| Temperature (° C.) | 550 | | 550 | |
| Collision Energy | 36 | 26 | 16 | 26 |
| Declustering Potential | 75 | 136 | 145 | 136 |

| | | |
|---|---|---|
| Entrance Potential | 10 | 10 |
| Collision Cell | 14 | 14 |
| Exit Potential | | |

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A compound having the structural formula (I), 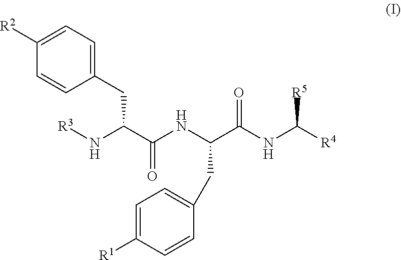
or a pharmaceutically acceptable form or an isotope derivative thereof,
wherein
R¹ is selected from the group consisting of H, F or Cl;
R² is selected from the group consisting of H, F or Cl;
R³ is selected from the group consisting of H, tert-butyloxycarbonyl (Boc) or 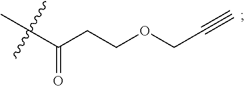 [SMILES: *C(=O)CCOCC#C]
R⁴ is 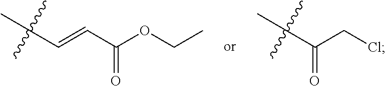
R⁵ is 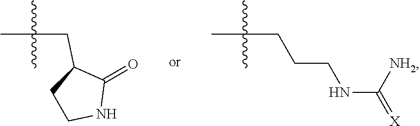
wherein X is O or NH₂⁺.

2. The compound of claim 1, wherein
R⁴ is 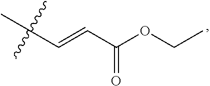 [SMILES: */C=C/C(=O)OCC]
and
R⁵ is 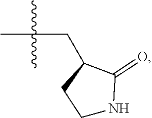 [SMILES: *C[C@@H]1CCNC1=O]
with the compound having the structural formula:

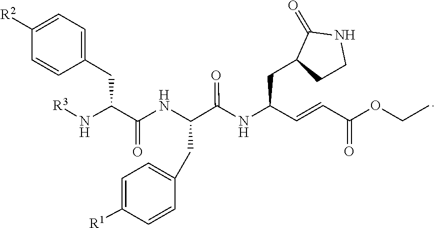

3. The compound of claim 2, wherein R¹ is H.

4. The compound of claim 2, wherein R¹ is F.

5. The compound of claim 2, wherein R² is H.

6. The compound of claim 2, wherein R² is F.

7. The compound of claim 2, wherein R³ is H.

8. The compound of claim 2, wherein R³ is Boc.

9. The compound of claim 1, wherein R³ is

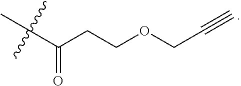 [SMILES: *C(=O)CCOCC#C]

10. A compound selected from the group consisting of:

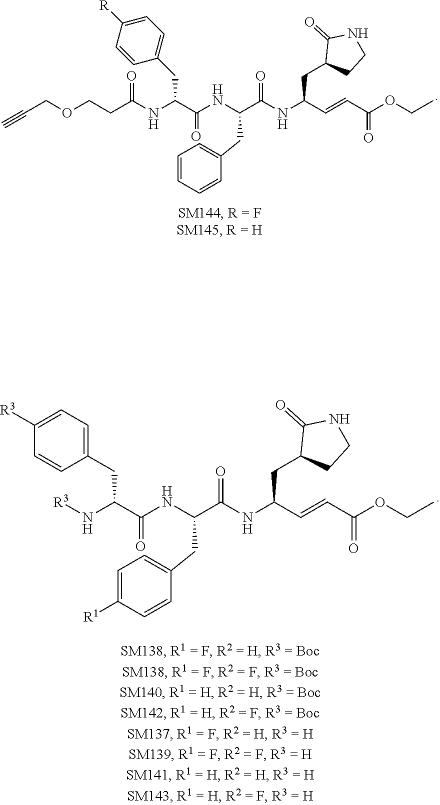

11. A pharmaceutical composition comprising a compound of claim 1.

12. A pharmaceutical composition comprising a compound having the structural formula of (I): 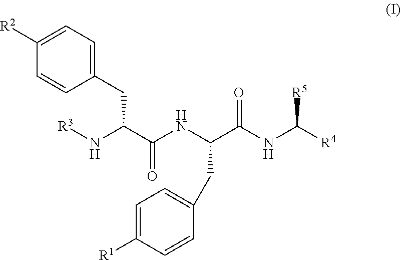
or a pharmaceutically acceptable form or an isotope derivative thereof,
wherein
R¹ is selected from the group consisting of H, F or Cl;
R² is selected from the group consisting of H, F or Cl;
R³ is selected from the group consisting of H, tert-butyloxycarbonyl (Boc) or 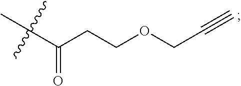 [SMILES: *C(=O)CCOCC#C]
R⁴ is 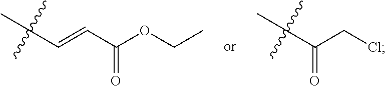
R⁵ is ON NH₂ 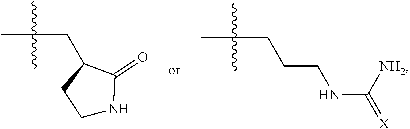
wherein X is O or NH₂⁺,
effective to treat, reduce or prevent one or more diseases or conditions, in a mammal, including a human, and a pharmaceutically acceptable excipient, carrier, or diluent.

13. The pharmaceutical composition of claim 11, wherein the one or more diseases or conditions is SARS-CoV2 or a related disease or condition.

14. A unit dosage form comprising a pharmaceutical composition according to claim 11.

15. The unit dosage form of claim 14, being in the form of a tablet or capsule.

16. A method for inhibiting or inactivating main protease (MPro) in a cell, comprising contacting the cell with a compound according to claim 1.

17. A method for inhibiting or inactivating an activity of main protease (MPro) in vitro or in vivo, comprising contacting the cell with a compound according to claim 1.

18. A method for treating, reducing or preventing a disease or condition, comprising administering to a subject in need thereof a compound according to claim 1.

19. (canceled)

20. The method of claim claim 18, wherein the one or more diseases or conditions is mediated by or associated with an activity of main protease (MPro).

21. The method of claim 20, wherein the one or more diseases or conditions is SARS-CoV2 or a related disease or condition.

22. (canceled)

23. (canceled)
